(19) United States
(12) Patent Application Publication
(10) Pub. No.: US 2009/0023182 A1
(43) Pub. Date: Jan. 22, 2009

(54) COMPLEMENTARY METABOLIZING ORGANISMS AND METHODS OF MAKING SAME

(76) Inventor: Christophe H. Schilling, San Diego, CA (US)

Correspondence Address: MCDERMOTT, WILL & EMERY, 4370 LA JOLLA VILLAGE DRIVE, SUITE 700, SAN DIEGO, CA, 92122, US

(21) Appl. No.: 11/779,865

(22) Filed: Jul. 18, 2007

(51) Int. Cl. C12P 39/00; C12N 1/00

(52) U.S. Cl. 435/42; 435/243

(57) ABSTRACT

The invention provides a non-naturally occurring set of microbial organisms. The set of organisms includes: at least a first constituent complementary metabolizing organism (CMO) exhibiting the ability to metabolize a first carbon substrate and having substantially impaired metabolic capacity for a second carbon substrate, and at least a second constituent complementary metabolizing organism (CMO) exhibiting the ability to metabolize the second carbon substrate and having substantially impaired metabolic capacity for the first carbon substrate, wherein a co-culture of the at least first and second CMOs exhibit simultaneous metabolism of a mixture having the first and second carbon substrates compared to either CMO alone. Simultaneous metabolism of a mixture having first and second carbon substrates can include an enhanced rate of metabolism of the first and second substrates compared to either CMO alone. Also provided is a bioprocess for producing a chemical compound. The bioprocess includes co-culturing a non-naturally occurring set of microbial organisms in a mixture having at least a first and a second carbon substrate under conditions sufficient for biosynthesis of a target chemical compound, the set of non-naturally occurring microbial organisms including: at least a first constituent complementary metabolizing organism (CMO) exhibiting the ability to metabolize the first carbon substrate and having substantially impaired metabolic capacity for the second carbon substrate, and at least a second constituent complementary metabolizing organism (CMO) exhibiting the ability to metabolize the second carbon substrate and having substantially impaired metabolic capacity for the first carbon substrate, wherein a co-culture of the at least first and second CMOs exhibit simultaneous metabolism of a mixture having the first and second carbon substrates compared to either CMO alone. Simultaneous metabolism of a mixture having first and second carbon substrates can include an enhanced rate of metabolism of the first and second substrates compared to either CMO alone.

### BACKGROUND OF THE INVENTION

This invention relates generally to the creation and engineering of organisms and, more particularly to organisms having complementary utilization of carbon sources.

Converting low cost renewable feedstocks into higher value chemical products using biological processes is desirable from both an economic and environmental standpoint. Carbohydrates such as glucose have served as a traditional feedstock for the fermentation-based production of a number of chemical products. Relative to non-renewable fossil fuel feedstocks for chemical processes, glucose has enjoyed significantly less price volatility over the past few decades. However, even lower cost renewable feedstocks are sought, as their use can lower the overall production costs, allowing bioprocess-derived products to enter the market place and compete more effectively against petrochemical-derived products.

Plant and plant-derived biomass material has received recent attention as one source for such a low cost feedstock. Biomass can undergo enzyme or chemical mediated hydrolysis to liberate substrates which can be further processed via biocatalysis to produce chemical products of interest. These substrates include mixtures of carbohydrates, as well as aromatic compounds and other unspecified products that are collectively derived from the cellulosic, hemicellulosic, and lignin portions of the biomass. The carbohydrates generated from the biomass are a rich mixture of 5 and 6 carbon sugars that include, for example, glucose, xylose, arabinose, galactose, mannose, and fructose. Cost effective biological-based processes (bioprocesses) that seek to utilize biomass as a feedstock (e.g., cellulosic ethanol) should be able to effectively consume each substrate to achieve desirable process yields and productivity levels. However, effective utilization of such mixed sugar feedstocks is currently a significant challenge in the field of industrial biotechnology.

High yields and productivities are largely a function of the efficiency of the conversion of the substrate to the product and the rate at which cells are able to ferment the carbon substrates present in the growth medium. Current processes attempt to use organisms that have the capacity to utilize each substrate in a mixture through native metabolic pathways or engineer these abilities into one organism. This approach is often complicated by various metabolic and regulatory barriers within the cell that favor the use of one substrate over another. As a result, carbon sources tend to be utilized sequentially instead of simultaneously, in a manner termed diauxic (or more generally, multi-auxic) growth. This disparity in the prioritization and use of one substrate over another can lead to extended fermentation times or even the incomplete (or low level) utilization of certain substrates, leading to decreasing rates and yields that compromise the overall process economics.

Some efforts have been made to alleviate the above problems by using co-cultures of different organisms to treat cellulosic biomass and metabolize the obtained sugars (Taniguchi and Tanaka, Adv Biochem Eng Biotechnol. 90:35-62 (2004)). Many of these studies utilized different yeast strains and their existing variants (Latif and Rajoka, Bioresour Technol. 77: 57-63 (2001); Chadha et al., Acta Microbiol Immunol Hung. 42: 71-75 (1995); Chadha et al., Acta Microbiol Immunol Hung. 42: 53-59 (1995); Olsson et al., Appl Biochem Biotechnol. 129-132: 117-29 (2006); Keating, et al., J Ind Microbiol Biotechnol. 31: 235-44 (2004)), yeasts with bacteria (Qian et al., Appl Biochem Biotechnol. 134: 273-84 (2006); Szambelan et al., Biotechnol Lett. 26: 845-48 (2004); Lynd, L. R., D. A. Hogsett, and G. Spieles, 1993, Trustees of Dartmouth College, Hanover (NH): USA), and different bacterial species including those belonging to the genus Clostridia (Demain et al., Microbiol Mol Biol Rev. 69: 124-54 (2005)). Despite these efforts, co-cultures of natural microorganisms failed to increase the consumption of carbon sources in heterogeneous mixtures of feedstocks to practical levels above that obtained utilizing a single carbon source. Some co-cultures required laborious feedstock and/or culture preparatory steps while others maintained serial consumption of sugars or were limited in the range of the sugars that could be utilized.

Thus, there exists a need for methods and organisms that are capable of metabolizing heterogeneous mixtures of feedstocks at high rates for the biosynthesis of desired products. The present invention satisfies this need and provides related advantages as well.

### SUMMARY OF THE INVENTION

The invention provides a non-naturally occurring set of microbial organisms. The set of organisms includes: at least a first constituent complementary metabolizing organism (CMO) exhibiting the ability to metabolize a first carbon substrate and having substantially impaired metabolic capacity for a second carbon substrate, and at least a second constituent complementary metabolizing organism (CMO) exhibiting the ability to metabolize the second carbon substrate and having substantially impaired metabolic capacity for the first carbon substrate, wherein a co-culture of the at least first and second CMOs exhibit simultaneous metabolism of a mixture having the first and second carbon substrates compared to either CMO alone. Simultaneous metabolism of a mixture having first and second carbon substrates can include an enhanced rate of metabolism of the first and second substrates compared to either CMO alone. Also provided is a bioprocess for producing a chemical compound. The bioprocess includes co-culturing a non-naturally occurring set of microbial organisms in a mixture having at least a first and a second carbon substrate under conditions sufficient for biosynthesis of a target chemical compound, the set of non-naturally occurring microbial organisms including: at least a first constituent complementary metabolizing organism (CMO) exhibiting the ability to metabolize the first carbon substrate and having substantially impaired metabolic capacity for the second carbon substrate, and at least a second constituent complementary metabolizing organism (CMO) exhibiting the ability to metabolize the second carbon substrate and having substantially impaired metabolic capacity for the first carbon substrate, wherein a co-culture of the at least first and second CMOs exhibit simultaneous metabolism of a mixture having the first and second carbon substrates compared to either CMO alone. Simultaneous metabolism of a mixture having first and second carbon substrates can include an enhanced rate of metabolism of the first and second substrates compared to either CMO alone.

### BRIEF DESCRIPTION OF THE DRAWINGS

FIG. 1 is a schematic showing an exemplary design and product production of complementary metabolizing organisms (CMO).

FIG. 2 shows a flow chart exemplifying the construction and evaluation of complementary metabolizing organisms.

FIG. 3 shows a Venn diagram representing the genes required for substrate A and substrate B to be metabolized in any given organism. Glucose and xylose are exemplified as substrates A and B, respectively. Each circle represents the metabolic genes (or reactions) in an organism that are used to metabolize each substrate. The overlapping regions represent genes (or reactions) used to metabolize both substrates. The genes (or reactions) represented in the non-overlapping regions are targets for elimination to construct complementary metabolizing organisms.

FIG. 4 shows the OD vs. time plots of a zwf-pgi CMO constituent strain that preferentially metabolizes xylose (squares) over glucose (diamonds).

FIG. 5 shows growth curves of a xylB CMO constituent strain that preferentially metabolizes glucose (diamonds) over xylose (squares).

FIG. 6 shows the xylose and glucose consumption characteristics when zwf-pgi constituent and xylB constituent complementary metabolizing organisms are grown in a co-culture (xylose (diamonds); glucose (squares)).

FIG. 7 shows the xylose (diamonds) and glucose (squares) consumption characteristics of parental, wild-type E. coli MG1655.

### DETAILED DESCRIPTION OF THE INVENTION

This invention is directed to the design and engineering of microbial organisms that effectively utilize low-cost feedstocks having heterogeneous mixtures of two or more substrates. The microbial organisms of the invention can be beneficially used in bioprocesses employing such mixed feedstocks to produce a wide range of chemical and biochemical products of interest. The methods and microbial organisms of the invention are generally applicable to any mixture of feedstocks.

In one embodiment, the invention is directed to a design and implementation procedure that produces microbial organisms capable of simultaneously consuming multiple carbon substrates in parallel, thus, relieving repression or other regulation of these organisms which otherwise direct sequential consumption of carbon sources. Microbial organisms of the same or different species can be modified to coexist such that the competition between them for the same substrate is eliminated. Microbial organisms are engineered to have modified metabolic pathways such that each uptake of competing substrates between two organisms is inhibited. When co-cultured each strain of the modified set is prevented from uptaking a substrate available in the growth medium that the other modified strain is able to consume and are thus, complementary metabolizers (CM or complementary metabolizing organisms (CMO)). By complementing each other's metabolic abilities, a co-culture of constituent CMO constituent strains can metabolize all target substrates at a higher rate compared to the unmodified strains alone or in co-culture.

In another embodiment, the invention is directed to the design and construction of a set of CMOs consisting of a pair of constituent strains. The set of strains were engineered in an E. coli background and consume in parallel both xylose and glucose when co-cultured. One constituent strain retains the ability to metabolize xylose and is inhibited from utilizing glucose through functional disruption of metabolic pathways specific for glucose utilization. The other constituent strain complements the xylose metabolizer in that it retains the ability to metabolize glucose but is deficient in xylose utilization.

As used herein, the term “non-naturally” when used in reference to a microbial organism or microorganism of the invention is intended to mean that the microbial organism has at least one genetic alteration not normally found in a naturally occurring strain of the referenced species, including wild-type strains of the referenced species. “Wild-type,” or grammatical equivalents thereof, refers to the common genotype or phenotype, or genotypes or phenotypes, of an organism as it is found in nature or in a standard laboratory stock for a given organism. Genetic alterations include, for example, a gene deletion or some other functional disruption of the genetic material. Genetic alterations also include modifications introducing expressible nucleic acids encoding metabolic polypeptides, other nucleic acid additions, nucleic acid deletions and/or other functional disruption of the microbial genetic material. Such modification include, for example, coding regions and functional fragments thereof, for heterologous, homologous or both heterologous and homologous polypeptides for the referenced species. Exemplary metabolic polypeptides include enzymes within a metabolic pathway or uptake pathway for one or more carbon sources used by a referenced microbial organism such as enzymes within the glycolysis or the pentose phosphate pathways.

A metabolic modification refers to a biochemical reaction or transport process that is altered from its naturally occurring state. Therefore, non-naturally occurring microorganisms have genetic modifications to nucleic acids encoding metabolic polypeptides or, functional fragments thereof. Exemplary metabolic modifications are described further below for E. coli as a representative microbial organism.

The term “isolated” when used in reference to a microbial organism is intended to refer to an organism that is substantially free of at least one component of the referenced microbial organism as it is found in nature. The term includes a microbial organism that is removed from some or all components as it is found in its natural environment. The term also includes a microbial organism that is removed from some or all components as the microbial organism is found in non-naturally occurring environments. Therefore, an isolated microbial organism is partly or completely separated from other substances as it is found in nature or as it is grown, stored or subsists in non-naturally occurring environments. Specific examples of isolated microbial organisms include partially pure microbial organism, substantially pure microbial organisms and microbial organisms cultured in a medium that is non-naturally occurring.

As used herein, the terms “microbial organism,” “microbe,” “microbial” or “microorganism” is intended to mean any organism that exists as a microscopic cell that is included within the domains of archaea, bacteria or eukarya. Therefore, the term is intended to encompass prokaryotic or eukaryotic cells or organisms having a microscopic size and includes bacteria, archaea and eubacteria of all species as well as eukaryotic microorganisms such as yeast and fungi. The term also includes cell cultures of any species that can be cultured for the production of a biochemical.

As used herein, the term “set” when used in reference to constituent strains of a complementary metabolizing organism is intended to mean a plurality of organisms or strains in co-culture. In this context, plurality means at least two. A set can have more than two constituent strains including, for example, 3, 4, 5, 6, 7, 8, 9, or 10 more organisms or strains which together make up the set. In general, a set of complementary metabolizing organisms will include a comparable number of organisms or strains as there are fuel substrates in a mixture of substrates.

Numerical modifiers such as the terms first, second, third, and fourth when used in reference to, for example, an organism, a constituent organism, a carbon substrate and the like, refer to different species thereof, unless explicitly stated to the contrary. For example, reference to a first and a second constituent complementary metabolizing organism means two organisms or strains that differ, in contrast to two identical organisms or strains. Similarly, reference to first, second, third and fourth organisms or strains means four different organisms or strains that each have at least one difference compared to the others.

As used herein, the terms “complementary metabolizing organism” or “complementary metabolizer” is intended to mean an organism that preferentially metabolizes a first substrate and has a reduced ability to metabolize a second substrate. Reduced ability includes substantially lower ability to metabolize a second substrate compared to the wild-type or parental organism. Reduced ability also includes an organism substantially lacking the ability to metabolize a substrate compared to the wild-type or parental organism. Substantially lower ability refers to a deficiency in the rate of substrate metabolism or uptake. Substantially lacking a metabolic ability refers to an organism that is deficient in at least one enzymatic, regulatory or transport reaction needed to metabolize or uptake the referenced substrate. Therefore, the term as it is used herein includes organisms that preferentially metabolize a first substrate and exhibits a lower rate of, or lacks the ability to, metabolize a second substrate. A specific example of a complementary metabolizing organism is a mutually exclusive metabolizer (MEM), which can, for example, metabolize substrate A at wild-type rates and lacks the ability to metabolize substrate B. A pair or set of complementary metabolizing organisms or complementary metabolizers refers to two or more organisms that exhibit reduced metabolic competition for two or more metabolic substrates, or are non-competitive with respect to metabolizing two or more metabolic substrates. The term “metabolism,” or grammatical equivalents thereof, when used in reference to a complementary metabolizing organism refers to both metabolic and substrate transport pathways.

As used herein, the term “constituent” when used in reference to a set of complementary metabolizing organisms is intended to mean one organism within the set. Together, different constituent complementary metabolizing organisms within a set exhibit the ability to simultaneously metabolize at least two substrates within a mixture. Simultaneous metabolism or consumption can occur at any point during co-culture and generally occurs, for example, throughout some or all of the exponential growth phase once each organism has exited their respective lag phases. In particularly useful embodiments, simultaneous metabolism of different substrates by co-culture of constituent organisms results in an enhanced rate of metabolism of the substrates compared to each constituent organism alone.

As used herein, the term “substantially” when used in reference to impaired metabolic ability is intended to mean that the referenced organism lacks any measurable ability to proliferate on a referenced substrate. Therefore, the term includes complete loss of the referenced metabolic capability as well as insufficient production of product to have a measurable affect on the organism's growth rate. The complete loss can be due to, for example, a genetic alteration where one or more metabolic pathways are functionally deficient in converting the substrate into a metabolic product or transport pathways are functionally deficient in transport or uptake of the substrate into the organisms. The genetic alteration can occur at any point along a metabolic or transport pathway.

As used herein, the term “metabolic capacity” is intended to refer to the ability of an organism to perform a metabolic function. An organism exhibits metabolic capacity for a substrate when it has the ability to use the referenced substrate for growth.

As used herein, the term “feedstock” refers to a substance used as a raw material in an industrial process. When used in reference to a culture of microbial organisms such as a fermentation process with cells, the term refers to the raw material used to supply a carbon or other energy source for the cells. A “renewable” feedstock refers to a renewable energy source such as material derived from living organisms or their metabolic byproducts including material derived from biomass, often consisting of underutilized components like chaff. Agricultural products specifically grown for use as renewable feedstocks include, for example, corn, soybeans and cotton, primarily in the United States; flaxseed and rapeseed, primarily in Europe; sugar cane in Brazil and palm oil in South-East Asia. Therefore, the term includes the array of carbohydrates, fats and proteins derived from agricultural or animal products across the planet.

As used herein, the term “biomass” is intended to mean any plant-derived organic matter. Biomass available for energy on a sustainable basis includes herbaceous and woody energy crops, agricultural food and feed crops, agricultural crop wastes and residues, wood wastes and residues, aquatic plants, and other waste materials including some municipal wastes. Biomass feedstock compositions, uses, analytical procedures and theoretical yields are readily available from the U.S. Department of Energy and can be found described, for example, at the URL 1.eere.energy.gov/biomass/information_resources.html, which includes a database describing more than 150 exemplary kinds of biomass sources. Exemplary types of biomasses that can be used as feedstocks in the methods of the invention include cellulosic biomass, hemicellulosic biomass and lignin feedstocks or portions of feedstocks. Such biomass feedstocks contain, for example, carbohydrate substrates useful as carbon sources such as glucose, xylose, arabinose, galactose, mannose, fructose and starch.

As used herein, the term “parental microbial organism” or grammatical equivalent thereof refers to an organism that can be changed to produce a non-naturally occurring constituent complementary metabolizing organism. Therefore, a parent microbial organism is the organism to be modified by a metabolic modification for producing a constituent complementary metabolizing organism. The term parent microbial organism, as used herein, both naturally occurring microbial organisms as well as non-naturally occurring microbial organisms. For example, a parent microbial organism can be a wild-type strain as found in nature or in a common laboratory stock, including various strains of bacteria, yeast and other microbes as described herein. A parent microbial organism also can be an organism that contains naturally occurring or recombinantly engineered genetic modifications including, for example, one or more metabolic modifications.

Therefore, a “progeny microbial organism” refers to a microbial organism that has a different genotype compared to the parent microbial organism from which it was produced. A different genotype can include, for example, addition, deletion or substitution of one or more nucleotide and/or nucleic acid sequences. Therefore, a progeny microbial organism includes all sizes of nucleic acid additions, deletions or substitutions ranging from a single nucleotide to complete coding, regulatory and/or gene region sequences.

As used herein, the term “target” when used in reference to the biosynthesis of a chemical compound is intended to refer to a specified product that is to be synthesized by a microbial organism of the invention. Therefore, the term refers to a predetermined chemical compound to be produced by a bioprocess of the invention.

As used herein, the term “exogenous” is intended to mean that the referenced molecule or the referenced activity is introduced into the host microbial organism. Therefore, the term as it is used in reference to expression of an encoding nucleic acid refers to introduction of the encoding nucleic acid in an expressible form into the microbial organism. When used in reference to a biosynthetic activity such as a metabolic or substrate transport activity, the term refers to an activity that is introduced into the host reference organism. The activity can be introduced into the reference host organism by, for example, introducing an encoding nucleic acid or nucleic acids sufficient to confer the referenced activity onto the organism. The source can be, for example, a homologous or heterologous encoding nucleic acid that expresses the referenced activity following introduction into the host microbial organism. Therefore, the term “endogenous” refers to a referenced molecule or activity that is present in the host. Similarly, the term endogenous when used in reference to expression of an encoding nucleic acid refers to expression of an encoding nucleic acid contained within the microbial organism. The term “heterologous” refers to a molecule or activity derived from a source other than the referenced species whereas “homologous” refers to a molecule or activity derived from the host microbial organism. Accordingly, exogenous expression of an encoding nucleic acid of the invention can utilize either or both a heterologous or endogenous encoding nucleic acid.

The non-naturally occurring microbal organisms of the invention can contain stable genetic alterations, which refers to microorganisms that can be cultured for greater than five generations without loss of the alteration. Generally, stable genetic alterations include modifications that persist greater than 10 generations, particularly stable modifications will persist more than about 25 generations, and more particularly, stable genetic modifications will be greater than 50 generations, including indefinitely.

Those skilled in the art will understand that the genetic alterations, including metabolic modifications exemplified herein are described with reference to E. coli genes and their corresponding metabolic reactions. However, given the complete genome sequencing of a wide variety of organisms and the high level of skill in the area of genomics, those skilled in the art will readily be able to apply the teachings and guidance provided herein to essentially all other organisms. For example, the E. coli metabolic alterations exemplified herein can readily be applied to other species by incorporating the same or analogous encoding nucleic acid from species other than the referenced species. Such genetic alterations include, for example, genetic alterations of species homologs, in general, and in particular, orthologs, paralogs or nonorthologous gene displacements.

An ortholog is a gene or genes that are related by vertical descent and are responsible for substantially the same or identical functions in different organisms. For example, mouse epoxide hydrolase and human epoxide hydrolase can be considered orthologs for the biological function of hydrolysis of epoxides. Genes are related by vertical descent when, for example, they share sequence similarity of sufficient amount to indicate they are homologous, or related by evolution from a common ancestor. Genes can also be considered orthologs if they share three-dimensional structure but not necessarily sequence similarity, of a sufficient amount to indicate that they have evolved from a common ancestor to the extent that the primary sequence similarity is not identifiable. Genes that are orthologous can encode proteins with sequence similarity of about 25% to 100% amino acid sequence identity. Genes encoding proteins sharing an amino acid similarity less that 25% can also be considered to have arisen by vertical descent if their three-dimensional structure also shows similarities. Members of the serine protease family of enzymes, including tissue plasminogen activator and elastase, are considered to have arisen by vertical descent from a common ancestor.

Orthologs include genes or their encoded gene products that through, for example, evolution, have diverged in structure or overall activity. For example, where one species encodes a gene product exhibiting two functions and where such functions have been separated into distinct genes in a second species, the three genes and their corresponding products are considered to be orthologs. For the growth-coupled production of a biochemical product, those skilled in the art will understand that the orthologous gene harboring the metabolic activity to be disrupted is to be chosen for construction of the non-naturally occurring microorganism. An example of orthologs exhibiting separable activities is where distinct activities have been separated into distinct gene products between two or more species or within a single species. A specific example is the separation of elastase proteolysis and plasminogen proteolysis, two types of serine protease activity, into distinct molecules as plasminogen activator and elastase. A second example is the separation of mycoplasma 5′-3′ exonuclease and Drosophila DNA polymerase III activity. The DNA polymerase from the first species can be considered an ortholog to either or both of the exonuclease or the polymerase from the second species and vice versa.

In contrast, paralogs are homologs related by, for example, duplication followed by evolutionary divergence and have similar or common, but not identical functions. Paralogs can originate or derive from, for example, the same species or from a different species. For example, microsomal epoxide hydrolase (epoxide hydrolase I) and soluble epoxide hydrolase (epoxide hydrolase II) can be considered paralogs because they represent two distinct enzymes, co-evolved from a common ancestor, that catalyze distinct reactions and have distinct functions in the same species. Paralogs are proteins from the same species with significant sequence similarity to each other suggesting that they are homologous, or related through co-evolution from a common ancestor. Groups of paralogous protein families include HipA homologs, luciferase genes, peptidases, and others.

A nonorthologous gene displacement is a nonorthologous gene from one species that can substitute for a referenced gene function in a different species. Substitution includes, for example, being able to perform substantially the same or a similar function in the species of origin compared to the referenced function in the different species. Although generally, a nonorthologous gene displacement will be identifiable as structurally related to a known gene encoding the referenced function, less structurally related but functionally similar genes and their corresponding gene products nevertheless will still fall within the meaning of the term as it is used herein. Functional similarity requires, for example, at least some structural similarity in the active site or binding region of a nonorthologous gene compared to a gene encoding the function sought to be substituted. Therefore, a nonorthologous gene includes, for example, a paralog or an unrelated gene.

Therefore, in identifying and constructing the non-naturally occurring microbial organisms of the invention having complementary metabolizing capability, given the teachings and guidances provided herein to a particular species those skilled in the art will understand that the identification of metabolic modifications can include identification and inclusion or inactivation of orthologs. To the extent that paralogs and/or nonorthologous gene displacements are present in the referenced microorganism that encode an enzyme catalyzing a similar or substantially similar metabolic reaction, those skilled in the art also can utilize these evolutionally related genes.

Orthologs, paralogs and nonorthologous gene displacements can be determined by methods well known to those skilled in the art. For example, inspection of nucleic acid or amino acid sequences for two polypeptides will reveal sequence identity and similarities between the compared sequences. Based on such similarities, one skilled in the art can determine if the similarity is sufficiently high to indicate the proteins are related through evolution from a common ancestor. Algorithms well known to those skilled in the art, such as Align, BLAST, Clustal W and others compare and determine a raw sequence similarity or identity, and also determine the presence or significance of gaps in the sequence which can be assigned a weight or score. Such algorithms also are known in the art and are similarly applicable for determining nucleotide sequence similarity or identity. Parameters for sufficient similarity to determine relatedness are computed based on well known methods for calculating statistical similarity, or the chance of finding a similar match in a random polypeptide, and the significance of the match determined. A computer comparison of two or more sequences can, if desired, also be optimized visually by those skilled in the art. Related gene products or proteins can be expected to have a high similarity, for example, 25% to 100% sequence identity. Proteins that are unrelated can have an identity which is essentially the same as would be expected to occur by chance, if a database of sufficient size is scanned (about 5%). Sequences between 5% and 24% may or may not represent sufficient homology to conclude that the compared sequences are related. Additional statistical analysis to determine the significance of such matches given the size of the data set can be carried out to determine the relevance of these sequences.

Exemplary parameters for determining relatedness of two or more sequences using the BLAST algorithm, for example, can be as set forth below. Briefly, amino acid sequence alignments can be performed using BLASTP version 2.0.8 (Jan. 5, 1999) and the following parameters: Matrix: 0 BLOSUM62; gap open: 11; gap extension: 1; x_dropoff: 50; expect: 10.0; wordsize: 3; filter: on. Nucleic acid sequence alignments can be performed using BLASTN version 2.0.6 (Sep. 16, 1998) and the following parameters: Match: 1; mismatch: −2; gap open: 5; gap extension: 2; x_dropoff: 50; expect: 10.0; wordsize: 11; filter: off. Those skilled in the art will know what modifications can be made to the above parameters to either increase or decrease the stringency of the comparison, for example, and determine the relatedness of two or more sequences.

The invention provides a non-naturally occurring set of microbial organisms. The set of organisms includes: at least a first constituent complementary metabolizing organism (CMO) exhibiting the ability to metabolize a first carbon substrate and having substantially impaired metabolic capacity for a second carbon substrate, and at least a second constituent complementary metabolizing organism (CMO) exhibiting the ability to metabolize the second carbon substrate and having substantially impaired metabolic capacity for the first carbon substrate, wherein a co-culture of the at least first and second CMOs exhibit simultaneous metabolism of a mixture including the first and second carbon substrates compared to either CMO alone. Simultaneous metabolism of a mixture having first and second carbon substrates can include an enhanced rate of metabolism of the first and second substrates compared to either CMO alone.

In some embodiments, the microbial organisms and methods of the invention circumvent regulatory controls of naturally occurring organisms by employing a heterogeneous population of modified microbial organisms designed to perform a specified bioconversion of mixed substrate media and/or feedstock. This heterogeneous population of microbial organisms can include differently engineered members of the same or different species. The effective use of microbial organisms in co-culture having the ability to utilize a substrate media having mixed carbon compounds has been a long-felt need and would be considered a breakthrough, high payoff opportunity in the conversion of biomass to useful products (Energy, Department of, Breaking the Biological Barriers to Cellulosic Ethanol. (2006)). The microbial organisms and methods of the invention therefore include differently engineered organisms specifically designed for to metabolize multiple different carbon sources in mixture. The microbial organisms of the invention also can be engineered to convert substrates into products in one integrated bioprocess, including one stage or sequential stage bioconversions.

In other embodiments, substrates can be derived from a variety of different biomasses. In yet other embodiments, hydrolysis of biomass can generate toxic compounds which also can be beneficially utilized from the substrate media as carbon sources for bioprocessing. Exemplary toxic compounds that can be harnessed as carbon or other fuel sources include furfurals, aromatics, acetate and other undetermined substrates. Removal of these toxic compounds also is particularly useful to the overall cost effectiveness of the process because it eliminates requirements for implementation of separate unit operations prior to, for example, the actual bioconversion step. When used as substrates, toxic compounds can be consumed, for example, before the main bioconversion takes place or concurrently in the same reaction vessel. One specific embodiment, achieves toxic product removal by conversion into cell matter or other products of interest.

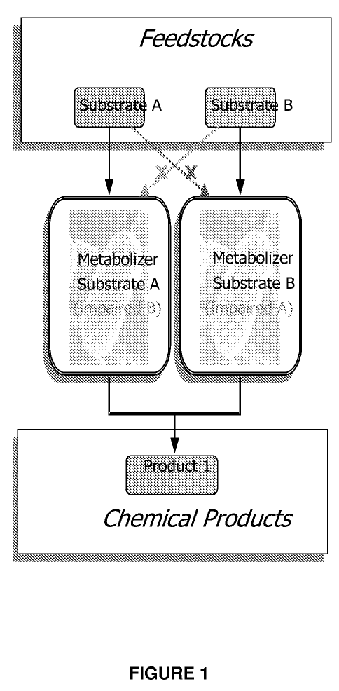

The microbial organisms of the invention are particularly useful because they allow for the complementary and simultaneous uptake of different carbon source substrates within a mixed population of organisms. Such mixed populations of organisms are engineered to preferentially and/or exclusively utilize different substrates in the media so that the differently engineered strains within the mixed population exhibit reduced metabolic competition or are substantially non-competitive with respect to metabolism of the same carbon source. Simultaneous and reduced competition for substrate uptake and utilization by the different strains also circumvents growth competition between species or strains in the co-culture. An exemplary embodiment of such complementary metabolizers (CM) of the invention is shown in FIG. 1. One organism within such a co-cultured population, CMO strain A, is designed and engineered to metabolize substrate A at the preferential exclusion of substrate B. The second organism in this specific example, CMO strain B, is designed and engineered to metabolize substrate B, also at the preferential exclusion of substrate A. When cultured together on a mixed substrate medium such as a feedstock, each strain will metabolize its substrate while together, both strains will utilize both substrates without competition from the other. As described further below, the above teachings and guidance apply equally to mixed co-cultures of more than two engineered CM organisms as well as to all organisms that can process or can be engineered to process multiple carbon substrates.

The non-naturally occurring sets of microbial organisms of the invention include organisms that employ metabolic modifications for the complementary metabolism of two or more energy sources during co-culture. Exemplary sets of such complementary metabolizing organisms (CMO) include a first constituent organism that metabolizes a first substrate and is substantially impaired in its ability to metabolize a second substrate. A complementary organism of the set includes a second constituent organism that metabolizes the second substrate, but is substantially impaired in its ability to metabolize the first substrate. The relationship of each constituent organism within a set is exemplified in FIG. 1, as described above.

A set of complementary metabolizing organisms includes at least two constituent organisms, each having the ability to metabolize alternative substrates within a mixture of substrates. Constituent organisms within a set complement the metabolic capabilities of the others within the set such that some or all substrates are utilized at a greater rate then they would be in a substrate mixture without a set of complementary metabolizing organisms of the invention. In particularly useful embodiments, the substrates within a mixture are simultaneously utilized by the constituent organisms. To achieve simultaneous consumption, each organism within the set should exhibit preferential metabolism or reduced metabolic competition of one substrate over other substrates within a mixture of substrates. Such simultaneous consumption can occur at any point throughout the growth phases of the constituent organisms including, for example, once each organism enters the growth phase as well as throughout some or all of the entire exponential growth phase. In other particularly useful embodiments, preferential metabolism includes substantially non-competitive metabolism of one substrate at the exclusion of all other substrates within a substrate mixture. As described further below, one method to arrive at such mutually exclusive metabolizers is to functionally disrupt metabolic pathway utilization for substrates other than the intended substrate.

A set of complementary metabolizing organisms includes a set of at least a first and a second constituent complementary metabolizing organism as described above. A set also can include three or more complementary metabolizing organisms that together are capable of simultaneously metabolizing three or more substrates contained within a mixture. For example, a first constituent CMO can exhibit the ability to metabolize a first substrate while having substantially impaired metabolic capacity for other substrates within the mixture, such as second and third different substrates. A second constituent CMO can exhibit the ability to metabolize a second substrate while having substantially impaired metabolic capacity for the first and third substrates. Similarly, a third constituent CMO can exhibit the ability to metabolize a third substrate which having substantially impaired metabolic capacity for the first and second substrates. Therefore, each of the first, second, third or more constituent CMO's will exhibit reduced metabolic competition or substantially non-competitive metabolism for their respective substrate compared to substrates metabolized by the other CMO's contained within the set.

Sets of complementary metabolizing organisms also can include a larger number of constituent CMOs to achieve the complementary metabolism of four, five or six or more different substrates within a mixture. In such sets of more than three constituent organisms the design and implementation follows the above exemplifications. For example, a set of complementary metabolizers for six different substrates includes a first constituent organism exhibiting the ability to metabolize a first substrate and having substantially impaired ability to metabolize substrates two through six. The second constituent organism should exhibit the ability to metabolize the second substrate and have substantially impaired ability to metabolize the first, third, fourth, fifth and sixth substrates. The remainder of the constituent organisms similarly will exhibit the ability to metabolize their respective substrates, but have substantially impaired metabolism for the remaining five substrates.

Given the teachings and guidance provided herein, those skilled in the art will understand that a set of complementary metabolizing organisms can encompass the ability to metabolize a wide range of different substrates. Therefore, the sets of complementary metabolizing organisms of the invention are applicable for use with a wide range of different substrate mixtures. A set generally includes, for example, a number of constituent complementary metabolizing organisms as there are substrates desired to be metabolize within a substrate mixture. However, sets can include metabolizers for some or all of the substrates within a mixture of different substrates.

Substrate mixtures include, for example, mixtures of sugars or other energy sources in growth media, fermentation broth or the like. For example, a set of complementary metabolizing organisms of the invention can be generated to grow on glucose and arabinose. A culture media can be obtained, produced or supplemented to contain both of these sugars. One constituent organism will preferentially utilize glucose while the complementary organism will preferentially utilize arabinose. Similarly, fermentation broth can be obtained, produced or supplemented to contain both of these sugars. Alternatively, heterogeneous mixtures having or capable of generating the requisite mixtures of energy sources also can be used as substrate mixture. A particular example of such a heterogeneous mixture includes a feedstock including, for example, renewable feedstocks and/or renewable feedstocks derived from biomass. Therefore, substrate mixtures include growth media, fermentation broth and/or complex feedstocks having two or more different energy sources. Other sources of substrate mixtures well known in the art also can be utilized with the sets of complementary metabolizing organisms of the invention. Following the teachings and guidance provided herein, those skilled in the art will understand such other sources can be utilized by generating a set of complementary metabolizing organisms capable of metabolizing together at least two or more energy sources contained therein.

Energy sources within a simple or complex mixture include, for example, carbohydrate, protein, lipid, fat and other macromolecules or chemical compounds applicable for conversion by cellular biochemical processes. Such energy sources typically supply the requisite carbon source for energy production used in biochemical process. Exemplary carbohydrates include, for example, simple and complex carbohydrates such as monosaccharides such as sugars and polysaccharides such as starches, respectively. Exemplary proteins include, for example, all types of polypeptides, including proteoglycans. These exemplary macromolecules as well as lipids, fats and other macromolecules are well known in the art and are all available as energy sources for the sets of complementary metabolizing organisms of the invention.

Exemplary materials and/or substances supplying these energy sources within complex mixtures such as biomass and/or renewable feedstocks include, for example, those described previously as well as other renewable resources or byproducts well known to those skilled in the art. For example, biomass can provide a wide variety of energy sources including the above carbohydrate, protein, lipid, fat as well as other molecules such as aromatic compounds and/or proteineaceous substances such as lignin. Biomass and renewable feedstocks are particularly useful as sources of a variety of carbohydrate. Such sources include, for example, cellulosic biomass, a hemicellulosic biomass, wheat straw, corn stover, reed canary grass, starch, corn, wheat or cotton woodchips starch, corn, wheat, cotton. Portions, chaff, fractions and waste products, for example, of these exemplary biomasses and renewable feedstocks as well as others well known in the art also are particularly useful sources for a variety of carbohydrates that can be used in a growth medium for a set of complementary metabolizing organisms of the invention.

Particularly useful substrate mixtures include medium or feedstocks containing different simple or complex carbohydrates. Carbohydrates provide an efficient carbon source for cellular proliferation. Exemplary carbohydrates include the sugars glucose, xylose, arabinose, galactose, mannose or fructose. Typically, a substrate mixture and set of microbial organisms will be matched or designed and generated to match such that the constituent organisms will complementary metabolize the carbon sources contained within the media. Thus, utilizing the exemplary sugars above, a complex mixture can contain, for example, glucose and xylose. The set of complementary metabolizing organisms will therefore have a first constituent preferentially metabolizing glucose and a second constituent preferentially metabolizing xylose. Similarly, this same set of complementary metabolizing organisms also can be used with a feedstock containing glucose and xylose to achieve simultaneous growth of each constituent. Other substrate mixtures comprising of two carbon sources can contain, for example, glucose and either arabinose, galactose and fructose. These exemplary substrate mixtures also can be formulated, for example, in a medium or broth or contained within a feedstock. Using the above sugars as for the purpose of illustration, it will be readily apparent to those skilled in the art that a set of complementary metabolizing organisms can be produced that simultaneously metabolize all combinations and permutations of the exemplified five sugars. These sets can be used with media or feedstock substrate mixtures containing the requisite combinations of sugars for the growth of all constitutive organisms within the set.

Feedstocks containing the sugar energy sources exemplified above or other carbon sources useful for growth of the complementary metabolizing organisms of the invention include, for example, cellulosic biomass, hemicellulosic biomass and lignin feedstocks. Such biomass feedstocks contain, for example, carbohydrate substrates useful as carbon sources such as glucose, xylose, arabinose, galactose, mannose, fructose and starch.

In addition to media containing know carbon substrates or other energy sources, the complementary metabolizing organisms of the invention can be designed and generated to utilize one or more byproducts, including toxic byproducts, generated during co-culture of the complementary metabolizing organisms. As described further below with respect to modifying a parent microbial organism to produce a constituent complementary metabolizing organism, one or more of parent or constituent organisms also can be modified to metabolize a byproduct of the culture or fermentation itself. In this specific embodiment, the initial at least two substrates contained in a medium supporting complementary metabolism of a co-cultured set of constituent organisms produces a renewable energy source that is further utilized by, for example, one or more of the initial constituent organisms or by an additional constituent organism capable of converting the byproduct into cellular energy sources such as ATP.

With exemplary reference to a set of two constituent complementary metabolizing organisms that complementarily metabolize two different substrates, each at the preferential exclusion of the other substrate, respectively, one or both constituent organisms of the set can be generated to further metabolize one or more byproducts. In this specific example, the first and second complementary metabolizing organisms exhibit the ability to metabolize first and second substrates, respectively, and further at least the first or second constituent exhibits the ability to metabolize the byproduct as a third substrate. Alternatively, a third constituent can be produced capable of metabolizing the third, byproduct substrate. Given the teachings and guidance provided herein, those skilled in the art will understand that the number of substrates that can be complementarily metabolized by a set of constituent organisms of the invention can be significantly increased by further utilization of byproducts of the culture. Accordingly, all combinations and permutations of first, second, third and fourth or more substrates and first, second, third and fourth or more byproducts within a co-culture grown on a substrate mixture can be utilized by producing at least two constituent organisms that complementarily metabolize, for example, one substrate, one byproduct or one substrate and one byproduct, while having impaired ability to metabolize the other substrates or byproducts within a substrate mixture and/or co-culture contain metabolic and/or substrate byproducts.

The byproduct can be, for example, any of the classes of energy sources described above that is released, produced or otherwise converted into a useable carbon source during culture. Alternatively, the byproduct can be a chemical compound that is generally toxic to microbial organisms of the invention. As described above, one or more constituent organisms can be engineered, for example, to preferentially metabolize the byproduct, including toxic byproduct, over or at the exclusion of other byproducts within the substrate mixture. Specific examples of toxic byproducts that can be utilized as energy sources include furfurals, aromatic compounds and acetate. For example, a constituent complementary metabolizing organism of the invention can be engineered to contain a metabolic modification conferring toluene dioxygenase and catechol 2,3-dioxygense activity. These activities can result in the production of pyruvate and/or acetaldehyde from an aromatic such as toluene following metabolism via a number of steps. Both pyruvate and acetaldehyde products can be utilized in central metabolic pathways.

Constituent complementary metabolizing organisms of the invention are designed and generated as described and exemplified above to utilize at least two carbon sources for energy production and/or cellular functions such as growth and biosynthesis. When co-cultured in a substrate mixture having the requisite at least two substrates the set of complementary metabolizing organisms exhibit simultaneous metabolism of the requisite substrates compared to each constituent complementary metabolism organism alone. In particularly useful embodiments, simultaneous metabolism of the requisite substrates results in an enhanced rate of metabolism of the requisite substrates compared to each constitutent metabolism organism alone. The enhanced rate of metabolism results from preferential metabolism via reduced metabolic competition or substantially non-competitive metabolism of each complementary metabolizing organism's cognate substrate without physiological or other regulatory controls, or without hindrances due to the presence of more than one substrate.

For example, a set of complementary metabolizing organisms made up of two constituent organisms that metabolize a first substrate A and a second substrate B will consume substrates A and B in parallel, resulting in an increased rate of substrate A and B utilization compared to either constituent organism alone. The increased rate of more than one energy source results in parallel proliferation and/or biosynthesis capabilities of all constituent organisms within the set which, in turn, can be harnessed for more efficient bioproduction of target compounds. These particularly useful characteristics result from uncoupling regulatory controls requiring sequential utilization of energy sources that are typically found in naturally occurring organisms or strains having unmodified metabolic and/or transport pathways for carbon source utilization. Parallel proliferation and biosynthesis capabilities also can be harnessed for more efficient consumption of byproducts, such as the removal or elimination of unwanted products, including waste products, within a substrate material. Similarly, a set of constituent organisms metabolizing first, second and third substrates A, B and C when co-cultured will utilize substrates A, B and C in parallel, also resulting in an increased rate of utilization of these substrates compared to either organism alone as well as increased proliferation and/or biosynthesis capabilities. The above specific embodiments are described with reference to sets of two or three complementary metabolizing organisms for purposes of illustration. However, given the teachings and guidance provided herein, those skilled in the art will understand that irrespective of the set size, the set of constituent organism will metabolize all ranges of substrates simultaneously and/or in parallel at an enhanced rate compared to the individual constituent organisms of the set.

In other embodiments, a set of complementary metabolizing organisms made up of two or more constituent organisms also will metabolize in parallel cognate energy sources in a substrate mixture simultaneously or at an enhanced rate during co-culture compared to a co-culture of parental microbial organisms. In further embodiments, a set of complementary metabolizing organisms of the invention also can be capable of metabolizing in parallel their cognate energy sources in a substrate mixture simultaneously or at an enhanced rate during co-culture compared to other non-related microbial organisms alone. Uncoupling of the constituent microbial organisms of the invention from cellular regulation requiring sequential utilization of energy sources will confer simultaneous and/or enhanced metabolic rates of substrate mixtures onto a set of complementary metabolizing organisms of the invention compared to parent, non-complementary metabolizing organisms in co-culture grown on the same substrate mixture or compared to a co-culture of other unrelated microbial organisms alone. Non-related microbial organisms include, for example, non-parent organisms as well as constituent complementary metabolizing organisms that are designed for metabolizing substrates other than the cognate energy sources of the referenced set of complementary metabolizing organisms.

The sets of complementary metabolizing organisms or constituent organisms thereof of the invention are described herein with general reference to energy source utilization, including carbon substrate or substrates utilization, metabolic reaction or pathway, reactant or product thereof, or with specific reference to one or more nucleic acids or genes encoding an enzyme associated with metabolizing a referenced energy source or catalyzing the referenced metabolic reaction, reactant or product. Unless otherwise expressly stated herein, those skilled in the art will understand that reference to a energy source also constitutes reference to the metabolic or transport pathway or pathways utilizing that energy source. Similarly, reference to a reaction also constitutes reference to the reactants and products of the reaction. Unless otherwise expressly stated herein, reference to an energy source or to a reactant or product also references the metabolic utilization pathway or pathways or the reaction, respectively; and that reference to any of these metabolic constitutes also references the gene or genes encoding the enzymes that catalyze the referenced reaction, reactant or product. Likewise, given the well known fields of metabolic biochemistry, enzymology and genomics, reference herein to a gene or an encoding nucleic acid also constitutes a reference to the corresponding encoded enzyme and the reaction it catalyzes as well as the reactants and products of the reaction.

As described previously, microbial organisms generally lack the capacity to metabolize substrate mixtures in parallel. In some embodiments, the invention contemplates circumvention of cellular regulation requiring sequential substrate utilization by utilizing at least microbial organisms unmodified with respect to carbon substrate utilization by using different microbial organisms that preferentially metabolize different initial substrates. Particularly useful embodiments of this aspect of the invention include use of at least one unmodified constituent organism together with a modified constituent organism where each constitutive member of the set exhibits substantially reduced metabolic competition or non-competitive metabolism of their respective substrates. A further embodiment includes use of at least two unmodified constituent organisms that exhibit substantially reduced metabolic competition or non-competitive metabolism of their respective substrates compared the substrate or substrates of the other constituent organisms within the set. For purposes of illustration, one unmodified organism that metabolizes a first glucose substrate, for example, prior to metabolizing any other substrate can be employed as a first constituent organism. A second unmodified organism that metabolizes a second fructose substrate, for example, prior to metabolizing any other substrate can be employed as a second constituent organism. Although the above organisms maintain sequential regulation of carbon substrate utilization, when combined into a set for growth on glucose and fructose each unmodified organism will metabolize its primary substrate over other substrates due to sequential regulation. In this illustrative example, the first organism will metabolize the first substrate glucose and the second organism will metabolize the second substrate fructose. Given the teachings and guidance provided herein, those skilled in the art will understand that a wide variety of naturally occurring organisms or common laboratory strains can be utilized as constituent organisms of the invention by combining at least two organisms having different primary metabolized substrates to circumvent normal sequential regulation and achieve elevated rates of a mixture of the primary substrates compared to either organism alone.

A particularly useful embodiment of the invention to generate a set of complementary metabolizing organisms by introducing metabolic modifications into parent organisms for the generation of essentially any desired constituent organism. Metabolic modifications that target functional disruption of one or more substrate utilization pathways and/or confer one or more desired substrate utilization pathways onto constituent organism can be used to circumvent sequential regulation substrate utilization occurring in most, if not all, microbial organisms. For example, a first constituent organism of a set of complementary metabolizing organisms can be designed and recombinantly engineered to metabolize any first carbon substrate and to impair any metabolic capacity for a second substrate. In like fashion, a second constituent organism of a set of complementary metabolizing organisms can be designed and recombinantly engineered to metabolize the second carbon substrate and to impair metabolic capacity for the first substrate. Following the teachings and guidance provided herein, any of the various sets of complementary metabolizing organisms exemplified previously can be generated to complementary metabolize a substrate mixture simultaneously and/or at enhanced rates by circumventing sequential substrate regulation of their parent organism or organisms.

Introduction of metabolic modifications into one or more parent organisms to generate constituent organisms is described further below with respect to the methods of the invention. A specific example of such metabolically modified constituent organisms also is described below in Example I, where a parent microbial organism able to metabolize glucose was modified to functionally disrupt xylose utilization, thus producing a glucose metabolizer constituent organism impaired in xylose utilization. A parent microbial organism able to metabolize xylose also was modified to disrupt glucose utilization, thus producing a xylose metabolizer constituent organism impaired in glucose utilization. In this specific example, the metabolic modifications were incorporated through functional disruptions of enzymes in the xylose and glucose metabolic pathways, respectively. In particular, a gene in each pathway was knocked out by recombinantly engineering a deletion of that target gene.

Exemplary parent microbial organisms can be selected from, and the constituent complementary metabolizing organisms generated in, for example, bacteria, yeast, fungus or any of a variety of other microorganisms applicable to fermentation processes. Exemplary bacteria include species selected from E. coli, A. succiniciproducens, A. succinogenes, M. succiniciproducens, R. etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicum, Pseudomonas fluorescens, and Pseudomonas putida. Exemplary yeasts include species selected from Saccharomyces cerevisiae, Schizosaccharomyces pombe, Kluyveromyces lactis, Kluyveromyces marxianus, Aspergillus terreus, Aspergillus niger and Pichia pastoris.

Those skilled in the art will understand that any molecular design and recombinant implementation, for example, can be used to add, delete or substitute one or more genes encoding enzymes in a metabolic substrate utilization pathway, including a metabolic and/or substrate transport pathway. For example, one or more constituent organisms within a set of complementary metabolizing organisms can be generated by, for example, utilizing an endogenous metabolic or transport pathway as exemplified above with respect to utilization of first and second substrates by the first and second constituent organisms, respectively. One or more constituent organisms within a set of complementary metabolizing organisms also can be generated by, for example, exogenously introducing one or more expressible nucleic acids encoding a metabolic or substrate transport pathway which confers a metabolic activity allowing the constituent organism to utilize a selected carbon substrate or other energy source. Similarly, endogenous or exogenously introduced deficiencies in a metabolic substrate utilization pathway also can be utilized or generated, respectively, for ensuring impaired ability to metabolize one or more substrates of a constituent organism. One exemplary metabolic modification that can be exogenously introduced to confer a specified metabolic ability for substrate utilization includes introducing the arabinose metabolism pathway into an arabinose deficient organism that has the pentose phosphate metabolism pathway to confer arabinose metabolic capabilities. The arabinose metabolic pathway can be generated by introducing expressible nucleic acids for the araA, araB and araD genes, which encode L-arabionose isomerase, L-ribulokinase and L-ribulose-phosphate 4-epimerase, respectively. Similarly, xylose metabolism can be conferred onto an organism by introducing expressible nucleic acids for xylA and xylB genes that encode xylose isomerase and xylulokinase, respectively.

In complementary fashion, one or more endogenous metabolic or transport pathways also can be modified by, for example, deletion or other functional inactivation of a gene encoding a metabolic or transport pathway polypeptide to generate a constituent organism having impaired metabolic capacity for a requisite second or first substrate. Using the example above for illustrative purposes, one or more constituent organisms within a set of complementary metabolizing organisms can be generated by, for example, introducing a metabolic modification such as a deletion, addition or substitution to knockout, mutate or inactivate one or more genes or their expression and/or regulatory elements that result in impaired metabolic capacity for the second and first substrates by the first and second constituent organisms, respectively. One or more constituent organisms within a set of complementary metabolizing organisms also can be generated by, for example, exogenously introducing one or more expressible nucleic acids, other than those described above introducing functional disruptions, which confer a metabolic or substrate pathway deficiency impairing the constituent organism's ability to metabolize a particular substrate. Such exogenously introduced deficiencies can include, for example, expressing one or more inhibitors or competitors of the targeted substrate metabolic pathway. Such inhibitors can be introduced to act, for example, at the gene level to prevent expression of a required gene and/or at the metabolic or transport pathway level to interfere with pathway function. By specific reference to two different carbon substrates, an exemplary metabolic modification which can be exogenously introduced to impair metabolic capacity of a constituent organism includes, for example, reducing or inhibiting mannose utilization. Lowering or preventing mannose utilization can be accomplished by, for example, reducing or inhibiting the activity of mannose-6-phosphate isomerase encoded by the manA gene using the methods described above such as mutating the endogenous manA gene to encode a non-functional polypeptide, mutating or otherwise disrupting expression or regulatory elements of the endogenous manA gene or deleting some or all of the manA gene or a manA expression or regulatory element. In a similar fashion, ribose assimilation can be reduced or inhibited by reducing or inhibiting ribokinase activity encoded by the rbsK gene as exemplified above.

Any set of constituent complementary metabolizing organisms of the invention also can be modified to generate a target chemical product such as a compound or polypeptide of interest. In this regard, one or both constituent organisms within a set of two complementary metabolizing organisms can be genetically modified to express one or more enzymes, or other polypeptides, that confer biosynthesis of a target compound. Similarly, one, two or more than two including all constituent organisms within a set of more than two complementary metabolizing organisms also can be genetically modified to express one or more enzymes, or other polypeptides, that confer biosynthesis of a target compound. For polypeptide products, one or more constituent organisms can be recombinantly engineered using methods well known in the art to express the gene or genes encoding the polypeptides of interest. For chemical compound biosynthesis, one or more constituent organisms also can be recombinantly engineered to express one or more enzymes conferring the desired metabolic modification onto the constituent host or hosts. Specific examples of such metabolic modifications include introducing genes directing the biosynthesis of, for example, ethanol, succinic acid, fumaric acid, an isoprenoid including amorphadiene and/or isopentenyl pyrophosphate, 3-hydroxypropionic acid 4-hydroxybutanoic acid and/or 1,4-butanediol.

Briefly, a constituent organism of the invention that can synthesize a chemical product is produced by ensuring that a host constituent organism includes functional capabilities for the complete biochemical synthesis of at least one target biosynthetic pathway. Ensuring at least one requisite target biosynthetic pathway confers the requisite biosynthesis capability onto the host constituent organism. For example, one or more constituent organisms of a set of complementary metabolizing organisms can be further engineered to include the requisite metabolic capabilities for succinic acid production. Genetic modifications conferring succinic acid production onto microbial organisms have been described in, for example, U.S. patent publication US2007/0111294 and include, for example, functional disruption of the genes within one of the following seven gene sets: (1) adhE, ldha; (2) adhE, ldhA, ackA-pta; (3) pfl, ldhA; (4) pfl, ldhA, adhE; (5) ackA-pta, pykF, atpF, sdhA; (6) ackA-pta, pykF, ptsG, or (7) ackA-pta, pykF, ptsG, adhE, ldhA, or an ortholog thereof. As described in US2007/0111294, a variety of additional genes also can be disrupted to achieve or augment succinic acid production.

One or more constituent organisms of a set of complementary metabolizing also can be further modified to include the requisite metabolic capabilities for production of fumaric acid. Genetic modifications conferring fumaric acid onto a microbial organism follow methods similar to those exemplified in US 2007/0111294 and include, for example, functional disruption of the genes within one of the following gene sets: (1) fumABC, zwf, purU, or (2) fumABC, zwf, glyA, or an ortholog thereof. A variety of additional genes also can be disrupted to achieve or augment fumaric acid production and include, for example, the additional disruption of at least one gene selected from ackA-pta, gdhA, pntAB or at lest one gene selected from ackA-pta, yibO, ythE for the fumABC, zwf, purU gene set. In one useful embodiment, each of the three genes from ackA-pta, gdhA, pntAB or ackA-pta, yibO, ythE can be functionally disrupted for the fumABC, zwf, purU gene set.

As another example, one or more constituent organisms of a set complementary metabolizing organisms also can be further modified to include the requisite metabolic capabilities for production of an isoprenoid such as amorphadiene or an amorphadiene precursor such as isopentenyl pyrophosphate (IPP). Genetic modifications conferring amorphadiene or IPP production onto a microbial organism also follow methods similar to those exemplified in US2007/0111294 and include, for example, functional disruption of the genes within one of the following four gene sets: (1) aceA, pps, pgi, glk and gcd; (2) aceA, pps, pgi, glk, idnK and gntK; (3) adhE, eda or edd, mdh and pntAB, or (d) adhE, glk, ldhA, pntAB and pps, or an ortholog thereof. A variety of additional genes also can be disrupted to achieve or augment amorphadiene or IPP production and include, for example, the additional disruption of at least one gene from folD, glyA, pflAB, pflCD, tdcE, thrB or thrC for the above first and second gene sets corresponding to aceA, pps, pgi, glk and gcd and aceA, pps, pgi, glk, idnK and gntK, respectively.

As a further example, one or more constituent organisms of a set complementary metabolizing organisms also can be modified to include the requisite metabolic capabilities for production 3-hydroxypropionic acid production. Genetic modifications conferring 3-hydroxypropionic acid production onto a microbial organism also follow similar methods to those exemplified in US2007/0111294 and include, for example, functional disruption of the genes in a microbial organism utilizing an anaerobic β-alanine 3-hydroxypropionic acid precursor pathway within one of the following seven gene sets: (1) adhE, ldhA, pta-ackA; (2) adhE, ldhA, frdABCD; (3) adhE, ldhA, frdABCD, ptsG; (4) adhE, ldhA, frdABCD, pntAB; (5) adhE, ldhA, fumA, fumB, fumc; (6) adhE, ldhA, fumA, fumB, fumC, pntAB; (7) pflAB, ldhA or (8) adhE, ldhA, pgi, or an ortholog thereof. Production of 3-hydroxypropionic acid also can be conferred by, for example, functional disruption of the genes in a microbial organism utilizing an aerobic glycerol 3-hydroxypropionic acid precursor pathway of one of the following six gene sets: (1) tpi, zwf; (2) tpi, ybhE; (3) tpi, gnd; (4) fpb, gapA; (5) pgi, edd or (6) pgi, eda, or an ortholog thereof. A further avenue for 3-hydroxypropionic acid production also can include, for example, functional disruption of the genes in a microbial organism utilizing a glycerate 3-hydroxypropionic acid precursor pathway of one of the following four genes or gene sets: (1) eno; (2) yibO; (3) eno, atpH or other atp subunit, or (4) yibO, atpH, or other atp subunit, or an ortholog thereof.

Other genes that can be disrupted to achieve or augment 3-hydroxypropionic acid production include, for example, further disruption of at least one gene selected from aceEF, ptsG or frdABCD for the pflAB, IdhA gene set in an organism utilizing an anaerobic β-alanine 3-hydroxypropionic acid precursor pathway; further disruption of at least one gene selected from glk or frdABCD for the adhE, IdhA, pgi gene set in an organism utilizing an anaerobic β-alanine 3-hydroxypropionic acid precursor pathway; further disruption of at least one gene selected from zwf, adhC, gcd, mgsA or deoC for the tpi, zwf; tpi, ybhE or tpi, gnd gene sets in an organism utilizing aerobic glycerol 3-hydroxypropionic acid precursor pathway; further disruption of at least one gene selected from glpX, gapC, adhC, mgsA, fsa, talC or gcd for the fpb, gapA gene set in an organism utilizing aerobic glycerol 3-hydroxypropionic acid precursor pathway; further disruption of at least one gene selected from adhC, gcd or deoC for the pgi, edd or pgi, eda gene sets in an organism utilizing aerobic glycerol 3-hydroxypropionic acid precursor pathway; further disruption of at least both genes eno and yibO for the eno or yibO single gene disruptions in an organism the utilizing a glycerate 3-hydroxypropionic acid precursor pathway, and/or further disruption of at least one gene selected from atpABCDEFGHI, aceEF, pflA, pflB, sucCD or sucAB, pta-ackA for the eno, atpH, or other atp subunit, or the yibO, atpH, or other atp subunit in an organism utilizing a glycerate 3-hydroxypropionic acid precursor pathway.

A constituent organism of the invention for biosynthesis of a target product can be produced by introducing expressible nucleic acids encoding one or more of the enzymes participating in one or requisite biosynthetic pathways. Depending on the host constitutive organism chosen for biosynthesis, nucleic acids for some or all of a particular target biosynthetic pathway can be expressed. For example, as an additional example to those exemplified above, one or more constituent organisms of a set of complementary metabolizing organisms also can be modified to include the requisite metabolic capabilities for production of 4-hydroxybutanoic acid (4-HB), a metabolically engineered biosynthetic precursor of 1,4-butanediol (BDO), and/or for the production of BDO. Genetic modifications conferring production of monomeric 4-HB onto microbial organisms include, for example, introduction of at least one exogenous nucleic acid encoding 4-hydroxybutanoate dehydrogenase, CoA-independent succinic semialdehyde dehydrogenase, succinyl-CoA synthetase, CoA-dependent succinic semialdehyde dehydrogenase, glutamate:succinic semialdehyde transaminase or glutamate decarboxylase. Such modifications also can include introduction of expressible nucleic acids encoding at least the two gene products 4-hydroxybutanoate dehydrogenase and CoA-independent succinic semialdehyde dehydrogenase; succinyl-CoA synthetase and CoA-dependent succinic semialdehyde dehydrogenase, or glutamate:succinic semialdehyde transaminase and glutamate decarboxylase. Genetic modifications conferring production of BDO onto a microbial organism can include, for example, one or more of the modifications described above for the production of 4-HB and the additional introduction of at least one exogenous nucleic acid encoding CoA-independent aldehyde dehydrogenase, CoA-dependent aldehyde dehydrogenase or alcohol dehydrogenase.

Given the teachings and guidance provided herein, those skilled in the art will understand that the number of encoding nucleic acids to introduce in an expressible form will parallel the target pathway deficiencies of the selected host constituent organism. Therefore, one or more constituent organisms of the invention can have one, two, three, four, five or six encoding nucleic acids encoding the enzymes constituting the target product biosynthetic pathway or pathways. In some embodiments, the constituent organisms also can include other genetic modifications that facilitate or optimize target product biosynthesis or that confer other useful functions onto the host microbial organism.

Sources of encoding nucleic acids for the various metabolic modifications or other recombinantly engineered modifications exemplified herein can include, for example, any species where the encoded gene product is capable of catalyzing the referenced reaction or activity. Such species include both prokaryotic and eukaryotic organisms including, but not limited to, bacteria, archaea, eubacteria, animal, mammal, including human. For example, the constituent complementary metabolizing organisms having target compound biosynthetic capability are exemplified herein with reference to E. coli hosts. However, with the complete genome sequence available now for more than 550 species (with more than half of these available on public databases such as the NCBI), including 395 microorganism genomes and a variety of yeast, fungi, plant, and mammalian genomes, the identification of genes encoding the requisite metabolic modification or other modification for one or more genes in related or distant species, including for example, homologs, orthologs, paralogs and nonorthologous gene displacements of known genes, and the interchange of genetic alterations between organisms is routine and well known in the art. Accordingly, the metabolic alterations enabling complementary metabolism of a substrate mixture by a set of constituent organisms or the biosynthesis of target compounds described herein with reference to a particular organism such as E. coli can be readily applied to other microorganisms, including prokaryotic and eukaryotic organisms alike. Given the teachings and guidance provided herein, those skilled in the art will know that a metabolic alteration exemplified in one organism can be applied equally to other organisms.

Methods for constructing and testing the expression levels of any of the non-naturally occurring constituent organisms, including those modified to synthesize a target compound of interest, can be performed, for example, by recombinant expression and detection methods well known in the art. Such methods can be found described in, for example, Sambrook et al., Molecular Cloning: A Laboratory Manual, Third Ed., Cold Spring Harbor Laboratory, New York (2001); Ausubel et al., Current Protocols in Molecular Biology, John Wiley and Sons, Baltimore, Md. (1999).

The non-naturally occurring constituent organisms of the invention are constructed using methods well known in the art as exemplified above to exogenously express at least one encoding nucleic acid in sufficient amounts to produce the referenced enzyme or transport protein for conferring the required substrate utilization or metabolic modification for target compound biosynthesis. Exemplary levels of expression for such exogenously introduced enzymes in each pathway are well known in the art In some embodiments, as described above, methods well known in the art are used to functionally disrupt an endogenous gene, and therefore, expression of the introduced nucleic acid is unnecessary.

Any of the above sets of complementary metabolizing organisms can be co-cultured under conditions sufficient for biosynthesis of the target chemical compound using methods well known to those skilled in the art. Generally, procedures for non-continuous culture, continuous culture and/or near-continuous culture for growth and/or production of a target product will include co-culturing a set of complementary metabolizing organisms of the invention in sufficient nutrients and medium to sustain and/or nearly sustain growth in an exponentially phase. Continuous culture under such conditions can include, for example, a day, 2, 3, 4, 5, 6 or 7 days or more. Additionally, continuous culture can include 1 week, 2, 3, 4 or 5 or more weeks and up to several months. It is to be understood that the continuous and/or near-continuous culture conditions also can include all time intervals in between these exemplary periods.

One particularly useful method for large scale bioproduction of a chemical product is fermentation. Briefly, fermentation procedures are well known in the art. Fermentation of a set of complementary metabolizing organisms in general, and for example, for the biosynthetic production of a target product of the invention such as a chemical compound can be utilized in, for example, batch fermentation, fed-batch fermentation; fed-batch fermentation or continuous fermentation. In addition, any of these methods of fermentation also can be coupled to well know separation methods applicable to fermentation procedures such as batch separation or continuous separation. Exemplary combinations of fermentation and separation methods applicable for bioproduction of a target chemical compound of the invention include, for example, batch fermentation and batch separation; batch fermentation and continuous separation; fed-batch fermentation and batch separation; fed-batch fermentation and continuous separation; continuous fermentation and batch separation or continuous fermentation and continuous separation.

Examples of batch and continuous fermentation procedures are well known in the art. An exemplary procedure for fed-batch fermentation and batch separation includes culturing a production organism such as a set of complementary metabolizing organisms in a 10 L bioreactor sparged with an N₂/CO₂ mixture, using 5 L broth containing 5 g/L potassium phosphate, 2.5 g/L ammonium chloride, 0.5 g/L magnesium sulfate, and 30 g/L corn steep liquor, and an initial first and second carbon source concentration of 20 g/L. As the CMOs grow and utilize the carbon sources, additional 70% carbon source mixture is fed into the bioreactor at a rate approximately balancing carbon source consumption. The temperature of the bioreactor is generally maintained at 30° C. Growth continues for approximately 24 hours or more, the target chemical compound reaches a concentration of between 20-200 g/L, with the cell density being between about 5 and 10 g/L. Upon completion of the cultivation period, the fermenter contents can be passed through a cell separation unit such as a centrifuge to remove cells and cell debris, and the fermentation broth can be transferred to a product separations unit. Isolation of the target chemical compound can take place by standard separations procedures well known in the art to separate organic products from dilute aqueous solutions, such as liquid-liquid extraction using a water immiscible organic solvent (e.g., toluene) to provide an organic solution of the target chemical compound. The resulting solution can then be subjected to standard distillation methods to remove and recycle the organic solvent and to isolate the target chemical compound having a known boiling point as a purified liquid, for example.

An exemplary procedure for continuous fermentation and continuous separation includes initially culturing a production organism such as a set of complementary metabolizing organisms in batch mode using, for example, a bioreactor apparatus and medium composition exemplified above, except that the initial at least first and second carbon source is about 30-50 g/L. When the carbon source is exhausted, feed medium of the same composition is supplied continuously at a rate of between about 0.5 L/hr and 1 L/hr, and liquid is withdrawn at the same rate. The target chemical compound concentration in the bioreactor generally remains constant at 30-40 g/L, and the cell density generally remains constant at between about 3-5 g/L. Temperature is generally maintained at 30° C., and the pH is generally maintained at about 4.5 using concentrated NaOH and HCl, as required. The bioreactor can be operated continuously, for example, for about one month, with samples taken every day or as needed to assure consistency of the target chemical compound concentration. In continuous mode, fermenter contents are constantly removed as new feed medium is supplied. The exit stream, containing cells, medium, and target chemical compounds or other desired products, can then be subjected to a continuous product separations procedure, with or without removing cells and cell debris, and can be performed by continuous separations methods well known in the art to separate organic products from dilute aqueous solutions and distillation and/or purifications methods such as those exemplified above and well known in the art.

In certain embodiments, the sets of complementary metabolizing organisms of the invention can be sustained, cultured or fermented under anaerobic or substantially anaerobic conditions. Briefly, anaerobic conditions refers to an environment devoid of oxygen. Substantially anaerobic conditions include, for example, a culture, batch fermentation or continuous fermentation such that the dissolved oxygen concentration in the medium remains between 0 and 10% of saturation. Substantially anaerobic conditions also includes growing or resting cells in liquid medium or on solid agar inside a sealed chamber maintained with an atmosphere of less than 1% oxygen. The percent of oxygen can be maintained by, for example, sparging the culture with an N₂/CO₂ mixture.

Therefore, the invention provides a bioprocess for producing a chemical compound. The bioprocess includes: co-culturing a non-naturally occurring set of microbial organisms in a mixture comprising at least a first and a second carbon substrate under conditions sufficient for biosynthesis of a target chemical compound, the set of non-naturally occurring microbial organisms having: (a) at least a first constituent complementary metabolizing organism (CMO) exhibiting the ability to metabolize the first carbon substrate and having substantially impaired metabolic capacity for the second carbon substrate, and (b) at least a second constituent complementary metabolizing organism (CMO) exhibiting the ability to metabolize the second carbon substrate and having substantially impaired metabolic capacity for the first carbon substrate, wherein a co-culture of the at least first and second CMOs exhibit simultaneous metabolism of a mixture comprising the first and second carbon substrates compared to either CMO alone.

The invention also provides a method of generating a set of complementary metabolizing organisms containing at least a first and second constituent complementary metabolizing organism as described above. The method includes identifying two or more energy sources for parallel growth of a set of complementary metabolizing organisms, identifying one or more metabolic modifications in at least one constituent complementary metabolizing organism conferring either the ability to metabolize a first substrate or conferring impaired metabolic capacity of a second substrate, and modifying one or more constituent complementary metabolizing organisms to incorporate the one or more metabolic modifications, wherein a set of constituent organisms is generated that includes: (a) at least a first constituent complementary metabolizing organism (CMO) exhibiting the ability to metabolize the first carbon substrate and having substantially impaired metabolic capacity for the second carbon substrate, and at least a second constituent complementary metabolizing organism (CMO) exhibiting the ability to metabolize the second carbon substrate and having substantially impaired metabolic capacity for the first carbon substrate, wherein a co-culture of the at least first and second CMOs exhibit simultaneous metabolism of a mixture including the first and second carbon substrates compared to either CMO alone. Simultaneous metabolism of a mixture having first and second carbon substrates can include an enhanced rate of metabolism of the first and second substrates compared to either CMO alone.

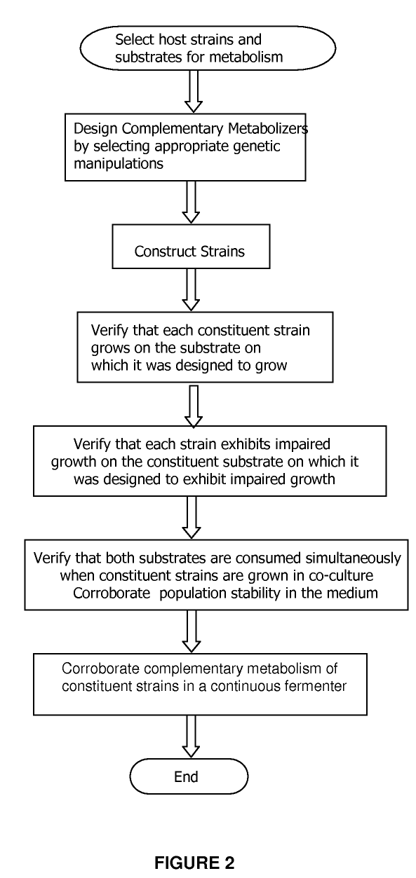

One general procedure for generating a set of complementary metabolizing organisms can begin with the identification of one or more required reactions that is desirable to eliminate and/or add to a parent organism's metabolic network in order to confer utilization of the first substrate of interest while not being able to consume the other substrates in a mixture. This step can be repeated for other substrates to generate a constituent organism design specifying the desired modifications. As described previously, these modifications to the organism will reduce or prevent competition for carbon substrates among the constituent organisms. Once the designs for desired metabolic modification and/or other genetic engineering are established, the constituent organisms are constructed using methods well known in the art as described above. Each constituent organism can be tested to corroborate that it can metabolize and grow on the select substrate while not being able to consume some or all of the other substrates in a mixture. The constituent organisms also can be corroborated for these same functions in a co-culture growing together in the same vessel with both design substrates present, thus confirming that the set is able to consume both the substrates without competing with one another. A schematic illustrating the flow of these exemplary steps is shown in FIG. 2.

One particularly useful method for identifying desirable reactions to eliminate, add or modify to generate the constituent organisms of the invention can focus on the import of substrates and delete the transporters for the substrate that that metabolic capacity is to be impaired. In following this approach, those skilled in the art will understand that it is beneficial to corroborate the specificity of a transporter targeted for deletion to ensure that its deletion also does not negate transport of a different desirable substrate. An alternative particularly useful method for identifying desirable reactions to eliminate, add or modify to generate the constituent organisms of the invention can focus on preventing the metabolism of the substrates through the removal of endogenous enzymatic reactions by deletion of the corresponding genes as described above.

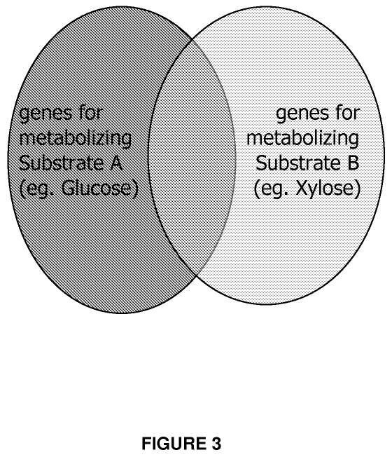

Briefly, FIG. 3 exemplifies the genes that are available for deletion in generating a constituent organism of the invention. With reference to the previously exemplified glucose and xylose set of complementary metabolizing organisms for purposes of illustration where glucose is a first substrate metabolized by a first constituent organism and xylose is a second substrate metabolized by a second constituent organism, then the genes that are utilized only for the metabolism of the individual sugars can be targeted for deletion. These subsets of genes are represented by the non-overlapping regions of glucose- and xylose-metabolizing genes in FIG. 3. In general, the first step that commits a substrate to metabolism is a particularly useful gene to eliminate. Such subsets of genes as well as those that commit a substrate to metabolism can be identified using, for example, a variety of methods well known in the art including, for example, computational metabolic models and/or metabolic models with the implementation of linear optimization algorithms as described below. Alternatively, such subsets also can be identified manually based on reports well known in the art or by empirical determination in a parent organism of interest. In selecting the gene or genes for functional disruption by, for example, gene deletion, those skilled in the art will understand that avoidance of genes required for other essential functions should be avoided.

One computational method for identifying and designing metabolic alterations favoring biosynthesis of a product is the OptKnock computational framework, Burgard et al., Biotechnol Bioeng, 84: 647-57 (2003). OptKnock is a metabolic modeling and simulation program that suggests gene deletion strategies that result in genetically stable microorganisms which overproduce the target product. Specifically, the framework examines the complete metabolic and/or biochemical network of a microorganism in order to suggest genetic manipulations that force the desired biochemical to become an obligatory byproduct of cell growth. By coupling biochemical production with cell growth through strategically placed gene deletions or other functional gene disruption, the growth selection pressures imposed on the engineered strains after long periods of time in a bioreactor lead to improvements in performance as a result of the compulsory growth-coupled biochemical production. Lastly, when gene deletions are constructed there is a negligible possibility of the designed strains reverting to their wild-type states because the genes selected by OptKnock are to be completely removed from the genome. Therefore, this computational methodology can be used to identify genes that can be disrupted to impair metabolic capacity of a substrate, to identify metabolic modifications that can be introduced to confer metabolic activity for a substrate as well as to identify pathways that lead to biosynthesis of target compound of interest.

Briefly, OptKnock is a term used herein to refer to a computational method and system for modeling cellular metabolism. The OptKnock program relates to a framework of models and methods that incorporate particular constraints into flux balance analysis (FBA) models. These constraints include, for example, qualitative kinetic information, qualitative regulatory information, and/or DNA microarray experimental data. OptKnock also computes solutions to various metabolic problems by, for example, tightening the flux boundaries derived through flux balance models and subsequently probing the performance limits of metabolic networks in the presence of gene additions or deletions. OptKnock computational framework allows the construction of model formulations that enable an effective query of the performance limits of metabolic networks and provides methods for solving the resulting mixed-integer linear programming problems. The metabolic modeling and simulation methods referred to herein as OptKnock are described in, for example, U.S. patent application Ser. No. 10/043,440, filed Jan. 10, 2002, and in International Patent No. PCT/US02/00660, filed Jan. 10, 2002.

Another computational method for identifying and designing metabolic alterations favoring biosynthetic production of a product is metabolic modeling and simulation system termed SimPheny®. This computational method and system is described in, for example, U.S. patent application Ser. No. 10/173,547, filed Jun. 14, 2002, and in International Patent Application No. PCT/US03/18838, filed Jun. 13, 2003.

SimPheny® is a computational system that can be used to produce a network model in silico and to simulate the flux of mass, energy or charge through the chemical reactions of a biological system to define a solution space that contains any and all possible functionalities of the chemical reactions in the system, thereby determining a range of allowed activities for the biological system. This approach is referred to as constraints-based modeling because the solution space is defined by constraints such as the known stoichiometry of the included reactions as well as reaction thermodynamic and capacity constraints associated with maximum fluxes through reactions. The space defined by these constraints can be interrogated to determine the phenotypic capabilities and behavior of the biological system or of its biochemical components. Analysis methods such as convex analysis, linear programming and the calculation of extreme pathways as described, for example, in Schilling et al., J. Theor. Biol. 203:229-248 (2000); Schilling et al., Biotech. Bioeng. 71:286-306 (2000) and Schilling et al., Biotech. Prog. 15:288-295 (1999), can be used to determine such phenotypic capabilities.

As described above, one constraints-based method used in the computational programs applicable to the invention is flux balance analysis. Flux balance analysis is based on flux balancing in a steady state condition and can be performed as described in, for example, Varma and Palsson, Biotech. Bioeng. 12:994-998 (1994). Flux balance approaches have been applied to reaction networks to simulate or predict systemic properties of, for example, adipocyte metabolism as described in Fell and Small, J. Biochem. 138:781-786 (1986), acetate secretion from E. coli under ATP maximization conditions as described in Majewski and Domach, Biotech. Bioeng. 35:732-738 (1990) or ethanol secretion by yeast as described in Vanrolleghem et al., Biotech. Prog. 12:434-448 (1996). Additionally, this approach can be used to predict or simulate the growth of E. coli on a variety of single-carbon sources as well as the metabolism of H. influenzae as described in Edwards and Palsson, Proc. Natl. Acad. Sci. 97:5528-5533 (2000), Edwards and Palsson, J. Bio. Chem. 274:17410-17416 (1999) and Edwards et al., Nature Biotech. 19:125-130 (2001).

Once the solution space has been defined, it can be analyzed to determine possible solutions under various conditions. This computational approach is consistent with biological realities because biological systems are flexible and can reach the same result in many different ways. Biological systems are designed through evolutionary mechanisms that have been restricted by fundamental constraints that all living systems must face. Therefore, constraints-based modeling strategy embraces these general realities. Further, the ability to continuously impose further restrictions on a network model via the tightening of constraints results in a reduction in the size of the solution space, thereby enhancing the precision with which physiological performance or phenotype can be predicted.

Given the teachings and guidance provided herein, those skilled in the art will be able to apply various computational frameworks for metabolic modeling and simulation to design and implement metabolic modifications to confer the requisite substrate utilization onto a constituent organism or to implement the biosynthesis of a target compound in a set of complementary metabolizing organisms of the invention. Such metabolic modeling and simulation methods include, for example, the computational systems exemplified above as SimPheny® and OptKnock. For illustration of the invention, some methods are described herein with reference to the OptKnock computation framework for modeling and simulation. Those skilled in the art will know how to apply the identification, design and implementation of the metabolic alterations using OptKnock to any of such other metabolic modeling and simulation computational frameworks and methods well known in the art.

The ability of a cell or organism to biosynthetically produce a biochemical product can be illustrated in the context of the biochemical production limits of a typical metabolic network calculated using an in silico model. These limits are obtained by fixing the uptake rate(s) of the limiting substrate(s) to their experimentally measured value(s) and calculating the maximum and minimum rates of biochemical production at each attainable level of growth. The production of a desired biochemical generally is in direct competition with biomass formation for intracellular resources. Under these circumstances, enhanced rates of biochemical production will necessarily result in sub-maximal growth rates. The knockouts suggested by the above metabolic modeling and simulation programs such as OptKnock are designed to restrict the allowable solution boundaries forcing a change in metabolic behavior from the wild-type strain. Although the actual solution boundaries for a given strain will expand or contract as the substrate uptake rate(s) increase or decrease, each experimental point will lie within its calculated solution boundary. Plots such as these enable accurate predictions of how close the designed strains are to their performance limits which also indicates how much room is available for improvement.

The OptKnock mathematical framework is exemplified herein for pinpointing gene deletions leading to product biosynthesis and, particularly, growth-coupled product biosynthesis. The procedure builds upon constraint-based metabolic modeling which narrows the range of possible phenotypes that a cellular system can display through the successive imposition of governing physico-chemical constraints, Price et al., Nat Rev Microbiol, 2: 886-97 (2004). As described above, constraint-based models and simulations are well known in the art and generally invoke the optimization of a particular cellular objective, subject to network stoichiometry, to suggest a likely flux distribution.

Briefly, the maximization of a cellular objective quantified as an aggregate reaction flux for a steady state metabolic network comprising a set N={1, . . . N} of metabolites and a set M={1, . . . , M} of metabolic reactions is expressed mathematically as follows:

$$\begin{matrix}maximize & {v}_{cellular objective} & \\ subject to & {\sum }_{j = 1}^{M} {S}_{ij} {v}_{j} = 0 , & ∀ i ∈ N \\ & {v}_{substrate} = {v}_{substrate uptake} m mol \text{/} g D W \cdot hr & ∀ i ∈ \{ limiting substrate ( s ) \} \\ & {v}_{atp} \geq {v}_{atp\_main} m mol \text{/} g D W \cdot hr & \\ & {v}_{j} \geq 0 , & ∀ j ∈ \{ irrev . reactions \}\end{matrix}$$

where Sᵢⱼ is the stoichiometric coefficient of metabolite i in reaction j, vⱼ is the flux of reaction j, V_{substrate}_{_{—}}ᵤₚₜₐₖₑ represents the assumed or measured uptake rate(s) of the limiting substrate(s), and Vₐₜₚ_{_{—}}^{main}, is the non-growth associated ATP maintenance requirement. The vector v includes both internal and external fluxes. In this study, the cellular objective is often assumed to be a drain of biosynthetic precursors in the ratios required for biomass formation, Neidhardt, F. C. et al., 2nd ed. 1996, Washington, D.C.: ASM Press. 2 v. (xx, 2822, 1xxvi). The fluxes are generally reported per 1 gDW·hr (gram of dry weight times hour) such that biomass formation is expressed as g biomass produced/gDW·hr or 1/hr.

The modeling of gene deletions, and thus reaction elimination, first employs the incorporation of binary variables into the constraint-based approach framework, Burgard et al., Biotechnol Bioeng, 74: 364-375 (2001), Burgard et al., Biotechnol Prog, 17: 791-797 (2001). These binary variables,

$${y}_{j} = \{ \begin{matrix}1 , if reaction flux {v}_{j} is active \\ 0 , if reaction flux {v}_{j} is not active\end{matrix} , ∀ j ∈ M$$

assume a value of 1 if reaction j is active and a value of 0 if it is inactive. The following constraint,

vⱼ^{min}·yⱼ≦v≦vⱼ^{max}∀jεM

ensures that reaction flux vⱼ is set to zero only if variable yⱼ is equal to zero. Alternatively, when yⱼ is equal to one, vⱼ is free to assume any value between a lower vⱼ^{min} and an upper vⱼ^{max} bound. Here, Vⱼ^{min} and vⱼ^{max} are identified by minimizing and maximizing, respectively, every reaction flux subject to the network constraints described above, Mahadevan et al., Metab Eng, 5: 264-76 (2003).

Optimal gene/reaction knockouts are identified by solving a bilevel optimization problem that chooses the set of active reactions (yⱼ=1) such that an optimal growth solution for the resulting network overproduces the chemical of interest. Mathematically, this bilevel optimization problem is expressed as the following bilevel mixed-integer optimization problem:

$${maximize}_{{y}_{j}} \qquad \qquad {v}_{chemical} \qquad \qquad \qquad \qquad \qquad \qquad \qquad \qquad \qquad \qquad \qquad \qquad ( OptKnock )$$

$$( \begin{matrix}{subject to}_{{v}_{j}} & maximize & {v}_{biomass} & \\ & subject to & {\sum }_{j = 1}^{M} {S}_{ij} {v}_{j} = 0 , & ∀ i ∈ N \\ & & {v}_{substrate} = {v}_{substrate\_uptake} & ∀ i ∈ \{ limiting substrate ( s ) \} \\ & & {v}_{atp} \geq {v}_{atp\_main} & \end{matrix} )$$

$${v}_{biomass} \geq {v}_{biomass}^{target}$$

$${v}_{j}^{min} \cdot {y}_{j} \leq {v}_{j} \leq {v}_{j}^{max} \cdot {y}_{j} , ∀ j ∈ M$$

$${\sum }_{j ∈ {M}^{forward}} ( 1 - {y}_{j} ) = K$$

$${y}_{j} ∈ \{ 0 , 1 \} , ∀ j ∈ M$$

where v_{chemical} is the production of the desired target product, for example succinate or other biochemical product, and K is the number of allowable knockouts. Note that setting K equal to zero returns the maximum biomass solution of the complete network, while setting K equal to one identifies the single gene/reaction knockout (yⱼ=0) such that the resulting network involves the maximum overproduction given its maximum biomass yield. The final constraint ensures that the resulting network meets a minimum biomass yield. Burgard et al., Biotechnol Bioeng, 84: 647-57 (2003), provide a more detailed description of the model formulation and solution procedure. Problems containing hundreds of binary variables can be solved in the order of minutes to hours using CPLEX 8.0, GAMS: The Solver Manuals. 2003: GAMS Development Corporation, accessed via the GAMS, Brooke et al., GAMS Development Corporation (1998), modeling environment on an IBM RS6000-270 workstation. The OptKnock framework has already been able to identify promising gene deletion strategies for biochemical overproduction, Burgard et al., Biotechnol Bioeng, 84: 647-57 (2003), Pharkya et al., Biotechnol Bioeng, 84: 887-899 (2003), and establishes a systematic framework that will naturally encompass future improvements in metabolic and regulatory modeling frameworks.

Any solution of the above described bilevel OptKnock problem will provide one set of metabolic reactions to disrupt. Because the reactions are known, a solution to the bilevel OptKnock problem also will provide the associated gene or genes encoding one or more enzymes that catalyze each reaction within the set of reactions. Identification of a set of reactions and their corresponding genes encoding the enzymes participating in each reaction is generally an automated process, accomplished through correlation of the reactions with a reaction database having a relationship between enzymes and encoding genes.

Once identified, the set of reactions that are to be disrupted in order to achieve target chemical production are implemented in the target cell or organism by functional disruption of at least one gene encoding each metabolic reaction within the set. One particularly useful means to achieve functional disruption of the reaction set is by deletion of each encoding gene. However, in some instances, it can be beneficial to disrupt the reaction by other genetic aberrations including, for example, mutation, deletion of regulatory regions such as promoters or cis binding sites for regulatory factors, or by truncation of the coding sequence at any of a number of locations. These latter aberrations, resulting in less than total deletion of the gene set can be useful, for example, when rapid assessments of the product coupling are desired or when genetic reversion is less likely to occur.

To identify additional productive solutions to the above described bilevel OptKnock problem which lead to further sets of reactions to disrupt or metabolic modifications that can result in the biosynthesis of biochemical products of interest, an optimization method, termed integer cuts, can be implemented. This method proceeds by iteratively solving the OptKnock problem exemplified above with the incorporation of an additional constraint referred to as an integer cut at each iteration. Integer cut constraints effectively prevent the solution procedure from choosing the exact same set of reactions identified in any previous iteration that obligatory couples product biosynthesis to growth. For example, if a previously identified growth-coupled metabolic modification specifies reactions 1, 2, and 3 for disruption, then the following constraint prevents the same reactions from being simultaneously considered in subsequent solutions: y₁+y₂+y_{3≧1}. The integer cut method is well known in the art and can be found described in, for example, reference, Burgard et al., Biotechnol Prog, 17: 791-797 (2001). As with all methods described herein with reference to their use in combination with the OptKnock computational framework for metabolic modeling and simulation, the integer cut method of reducing redundancy in iterative computational analysis also can be applied with other computational frameworks well known in the art including, for example, SimPheny®.

Constraints of the above form preclude identification of larger reaction sets that include previously identified sets. For example, employing the integer cut optimization method above in a further iteration would preclude identifying a quadruple reaction set that specified reactions 1, 2, and 3 for disruption since these reactions had been previously identified. To ensure identification of all possible reaction sets leading to biosynthetic production of a product, a modification of the integer cut method can be employed.

Briefly, the modified integer cut procedure begins with iteration ‘zero’ which calculates the maximum production of the desired biochemical at optimal growth for a wild-type network. This calculation corresponds to an OptKnock solution with K equaling 0. Next, single knockouts are considered and the two parameter sets, objstoreᵢₜₑᵣ and ystore_{iter,j}, are introduced to store the objective function (v_{chemical}) and reaction on-off information (yⱼ), respectively, at each iteration, iter. The following constraints are then successively added to the OptKnock formulation at each iteration.

v_{chemical}≧objstoreᵢₜₑᵣ+ε−M·Σ_{jεystore}_{_{iter,j}}₌₀yⱼ

In the above equation, ε and M are a small and a large numbers, respectively. In general, ε can be set at about 0.01 and M can be set at about 1000. However, numbers smaller and/or larger then these numbers also can be used. M ensures that the constraint can be binding only for previously identified knockout strategies, while censures that adding knockouts to a previously identified strategy must lead to an increase of at least ε in biochemical production at optimal growth. The approach moves onto double deletions whenever a single deletion strategy fails to improve upon the wild-type strain. Triple deletions are then considered when no double deletion strategy improves upon the wild-type strain, and so on. The end result is a ranked list, represented as desired biochemical production at optimal growth, of distinct deletion strategies that differ from each other by at least one knockout. This optimization procedure as well as the identification of a wide variety of reaction sets that, when disrupted, lead to the biosynthesis, including growth-coupled production, of a biochemical product. Given the teachings and guidance provided herein, those skilled in the art will understand that the methods and metabolic engineering designs exemplified herein are equally applicable to identify new biosynthetic pathways and/or to the obligatory coupling of cell or microorganism growth to any biochemical product.

The methods exemplified above and further illustrated in the Examples below enable the construction of constituent organisms for producing a set of complementary metabolizing organisms that can metabolize different substrates simultaneously and/or at enhanced rates. These methods also enable the construction of such constituent organisms that can further biosynthetically produce, including obligatory couple growth, to the production of a target chemical compound engineered to harbor the identified genetic alterations.

Therefore, the computational methods described herein enable the identification and implementation of metabolic modifications that are identified by an in silico method selected from OptKnock or SimPheny. The set of metabolic modifications can include, for example, addition of one or more biosynthetic pathway enzymes and/or functional disruption of one or more metabolic reactions including, for example, disruption by gene deletion.

It is understood that modifications which do not substantially affect the activity of the various embodiments of this invention are also included within the definition of the invention provided herein. Accordingly, the following examples are intended to illustrate but not limit the present invention.

### EXAMPLE I

### Complementary Metabolizing Organisms for Glucose and Xylose

This Example describes the design and construction of a complementary metabolizing (CM) set of bacterial strains that simultaneously metabolize a mixture of glucose and xylose in co-culture as compared to an unmodified or wild type strain.

Glucose and xylose were selected as a pair of substrates for demonstrating complementary metabolizing sets because they are both predominant sugars in cellulosic biomass, a desired low cost feedstock for bioprocesses. E. coli also was selected as the host organism to confer preferential substrate utilization in each of two engineered strains because it can naturally metabolize both glucose and xylose, albeit sequentially. Additionally, the genetic manipulation and culturing of this organism is very well characterized.

The set of CMO strain designs for utilization of a mixed carbon source of glucose and xylose consisted of one constituent strain engineered to metabolize glucose with little, if any, metabolism of xylose. The second constituent strain was designed and engineered to metabolize xylose with little, if any, metabolism of glucose. These constituents were designed by first determining the essential genes for each metabolic pathway or pathways utilizing glucose and xylose. The glucose constituent strain was generated by maintaining genes essential for glucose utilization and disrupting one or more genes required for xylose utilization. Similarly, the xylose constituent strain was generated by maintaining the essential genes for xylose utilization and disrupting one or more genes required for glucose utilization. Disruption of gene function was performed by gene deletion as described below.

Gene deletions for functional disruption of either glucose or xylose metabolism were identified using in silico modeling, from literature reports or both as described below. Beginning with xylose, it was determined that either xylA, encoding xylose isomerase, or xylB, encoding xylulokinase, should be deleted to prevent xylose metabolism in E. coli. Similarly, pgi and zwf, encoding phosphoglucoisomerase and glucose-6-phosphate dehydrogenase, respectively, were identified as the deletion targets for preventing glucose metabolism. The constituent strain genotype design for the glucose metabolizer was E. coli (AxylA or AxylB) whereas the xylose metabolizer had a genotype design of E. coli (Δpgi and Δzwf).

Briefly, an in silico stoichiometric model of E. coli metabolism was employed to identify essential genes for glucose and xylose metabolism as exemplified previously and described in, for example, U.S. patent publications US 2002/0012939, US 2003/0224363, US 2004/0029149, US 2004/0072723, US 2003/0059792, US 2002/0168654 and US 2004/0009466, and in U.S. Pat. No. 7,127,379. The in silico model determined that glucose metabolism can take place through glycolysis and the pentose phosphate pathway as well as through a route that converts glucose to gluconate, bypassing glucose-6-phosphate dehydrogenase. In this latter pathway, gluconate is subsequently channeled into the Entner Doudoroff pathway. The gluconate pathway is activated on glucose only in the presence of the precursor PQQ (Pyrroloquinoline quinone; Adamowicz et al., Appl Environ Microbiol, 57: 2012-15 (1991)). This precursor is not synthesized by E. coli and has to be provided in the medium if the pathway has to be activated. Therefore, this pathway is not naturally active on glucose and was ignored in the design and construction of the constituent glucose metabolizer.

Further, the model predicted that a pgi-zwf double deletion would lack the capability to grow on xylose because of its inability to produce g6p (glucose 6-phosphate), which in turn is needed for glycogen production. However, previous reports have described the viability of this strain on xylose (Fraenkel, D. G., J Biol. Chem., 243: 6451-57 (1968)). This apparent discrepancy was attributed to the inclusion of glycogen as an essential component of biomass in the in silico metabolic model of E. coli. Therefore, the pgi-zwf double deletion strain was selected as the second constituent strain of the glucose/xylose CMO set.

The above CMO constituent strain designs were generated with the assistance of a genome-scale in silico metabolic model of E. coli. However, empirical methods of constructing and testing candidate designs also can be employed for the design and construction of CMO constituent strains. An in silico model can therefore serve as one method to corroborate the consistency of the manually derived designs.

Each of the two CMO constituent strains described above were constructed and characterized for growth on glucose, xylose or both as described below. The zwf-pgi double deletion was constructed from Escherichia coli K-12 MG1655. Briefly, knockout deletions were integrated sequentially into the recipient strain employing in-frame deletions by homologous recombination via the λ Red recombinase system of Datsenko and Wanner (Proc. Nat. Acad. Sci. USA. 97:6640-45 (2000)). No drug resistance markers remained after each deletion, allowing multiple mutations to be accumulated in the target strains. In addition, complete removal of the targeted gene avoids the possibility of the constructed mutants reverting back to their wild-type. A xylB mutant strain was acquired from the KEIO collection of E. coli mutants (Baba et al., Mol Syst Biol. 2:2006-08 (2006).

The above constituent CMO strains were characterized by measuring their growth rates separately for both glucose and xylose carbon sources. Alternative methods of characterization include measuring the substrate uptake rates and/or the product or byproduct secretion rates. The measurements were performed separately for each strain. Measurements of a co-culture of each strain were also performed.

Briefly, cultures of each constituent strain were grown overnight and used as inoculum for a fresh batch culture for which measurements were taken during exponential growth. The growth rate was determined by measuring optical density using a spectrophotometer (A600). Concentrations of xylose in the culture supernatant were determined by HPLC using an HPX-87H column (BioRad) and those of glucose measured by enzymatic assay. These concentrations are then used to calculate uptake rates. Concentrations of ethanol and other byproducts in the culture supernatant are determined by HPLC as above and used to calculate secretion rates. All studies were performed with shake-flask cultures with volumes on the order of 25 mL or more.

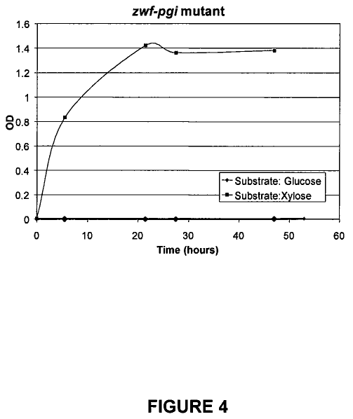

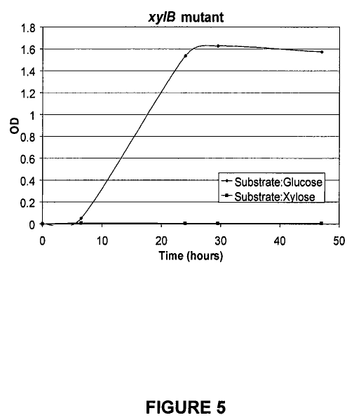

The xylose zwf-pgi metabolizing strain was grown separately on xylose and glucose. FIG. 4 shows the plotted growth curves of this strain on both substrates. The results demonstrate that this strain can proliferate on xylose. No measurable growth was observed on glucose. Similarly, the glucose xylB metabolizing strain was grown on glucose and xylose to corroborate that it could grow on glucose, the substrate on which it was designed to utilize, and not on xylose. The growth results of this strain are shown in FIG. 5 and demonstrate saturable growth on glucose with little, if any, measurable growth on xylose. These results show that the designed strains exhibit the required substrate uptake characteristics for complementary metabolizers. Corroboration that these strains do not compete with each other for the carbon substrates is described below.

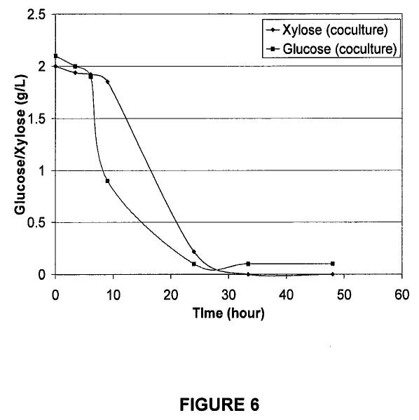

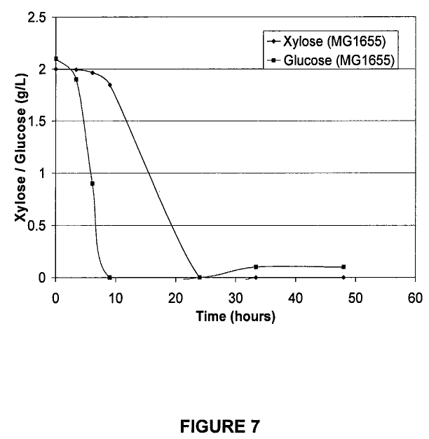

Briefly, simultaneously utilization of glucose and xylose by the constituent CMO strains described above also was assessed. The simultaneous growth of both the mutant strains was verified by co-culturing the glucose-producing constituent CMO and the xylose-producing constituent CMO in a 25 ml shake flask culture in M9 mineral media supplemented with 2 g/L each of glucose and xylose. The co-culture was inoculated with zwf-pgi constituent CMO (and the xylB constituent CMO such that the initial ODs of both strains were the same in the shake flask culture. The results are shown in FIGS. 6 and 7. FIG. 6 demonstrates that the co-cultured constituent CMO strains consumed both xylose and glucose simultaneously as compared to the sequential consumption of both the sugars by the parent, wild type strain MG1655 shown in FIG. 7. In this regard, FIG. 6 shows that the xylose consumption curve starts decreasing before the glucose substrate is completely consumed. In contrast, FIG. 7 shows that xylose does not start becoming consumed until after all of the glucose is utilized. The slightly increased utilization of both carbon sources by the parental E. coli strain compared to the co-culture of zwf-pgi constituent (xylose CMO; diamonds) and xylB (glucose CMO; squares) constituent CMOs can likely be explained by the relatively low growth rate of the zwf-pgi constituent CMO. It also is likely that this constituent strain can be evolved to have higher growth rates to achieve a comparable or enhanced overall sugar consumption rate of the sugars by the co-culture of constituent CMOs compared to the parental organism.

These results indicate that a co-culture of the xylose and glucose metabolizing strains can serve as complementary metabolizers to expedite the rate of consumption of both the substrates compared to unmodified or wild-type E. coli. This parallel consumption by CMO constituent strains, as compared to sequential consumption of wild-type strains, allows for a concomitant increase in desired product formation when used as a bioreactor.

Adaptive evolution also can be performed on one or both strains within the CMO set. For example, following deletion of any of the zwf, pgi or xylB genes, the growth or product synthesis characteristics can initially be less than predicted. Adjustment of the strains to their missing functionalities and achievement of predicted growth rates, product formation and/or both can be facilitated or achieved by adaptive evolution. As described previously, this process imposes growth as a selection pressure to compel the altered strains to reallocate their fluxes for enhancing growth rates. This reprogramming of metabolism has been recently demonstrated for several E. coli mutants that had been adaptively evolved on various substrates to reach the growth rates predicted a priori by an in silico model. Following adaptive evolution procedures, if any, each strain can be reassessed to corroborate that it maintained a xylose or glucose metabolizing CMO phenotype in a substantially exclusive fashion, and also in co-cultures with both sugars.

Parallel consumption of mixed substrates by CMO constituent strains also is evaluated in continuous culture during fermentation in a chemostat. Such parallel consumption with balanced growth of each constituent strain is particularly useful in a wide variety of bioprocesses for the production of chemical and biochemical products on mixed feedstocks.

Briefly, to counter the problem of washouts of the slow growing strain in chemostats, the dilution rates and the substrate uptake rates are adjusted as described below. In this specific study, the xylose strains are anticipated to grow at a slower rate than the glucose consuming strains. According to the simulations performed using the in silico platform model SimPheny®, the maximum growth rate with for an uptake rate of 10 mmol/gDW·hr of xylose is 0.69 per hour. The growth rate is slightly higher at 0.84 per hour when 10 mmol/gDW·hr of glucose is uptaken. Both of these rates are reported for substrate metabolisms in an aerobic growth environment. Under anaerobic conditions, the growth rates were determined to be 0.15 per hour for an uptake of 10 mmol/gDW.hr of xylose as compared to 0.21 per hour for the same rate of glucose uptake.

The optimum dilution rate for maximum productivity in a chemostat is calculated in the following manner:

$$\frac{d}{d D} ( D X ) = 0 ; \qquad ( 1 )$$

where D is the dilution rate (per hour) and X is the biomass yield (g/L)

$$or , \frac{d}{d D} ( D {Y}_{X / S} ( {S}_{0} - S ) ) = 0 ; \qquad ( 2 )$$

Y_{x/s} is the biomass yield per unit of substrate uptake. Sₒ corresponds to the initial substrate concentration and S refers to the substrate concentration.
For continuous fermentations,

$$D = \frac{{μ}_{max} \cdot S}{{K}_{S} + S}$$

(derived from the Michaelis-Menten equation). Therefore,

$$S = \frac{D \cdot {K}_{S}}{{μ}_{max} - D} .$$

Equation (2) then becomes

$$\frac{d}{d D} [ D \cdot {Y}_{X / S} \cdot ( {S}_{0} - ( \frac{D \cdot {K}_{S}}{{μ}_{max} - D} ) ) ] = 0 ;$$

The optimum dilution rate, Dₒₚₜ, can then be calculated as:

$${D}_{opt} = {μ}_{max} \cdot [ 1 - {( \frac{{K}_{S}}{{K}_{S} + {S}_{0}} )}^{1 / 2} ] \qquad ( 3 )$$

where μₘₐₓ and Kₛ are parameters that are acquired from literature. Dₒₚₜ is a function of the initial substrate concentration; therefore, the feed rates for xylose and glucose are adjusted such that one value of Dₒₚₜ for both the substrates is obtained. The calculated value of the dilution rate is lower than the washout rates for both glucose and xylose.

Following the above procedure, the constituent strains with the best xylose and glucose uptake characteristics and satisfactory correlation to the in silico model predictions are grown in a chemostat for one month to confirm long-term stability in carbon source utilization. The chemostat cultivation is performed using M9 minimal media supplemented with the requisite carbon substrates in a 1.3-L benchtop fermenter (New Brunswick Scientific, Edison, N.J.) at a working volume of approximately 600 mL. Carbon source concentrations and the dilution rate are adjusted at the values calculated according to equation (3) above. Sterile air is used for aerobic growth, and the dissolved oxygen is maintained at >95% of saturation using the agitation rate. Metabolic behavior of the co-culture is evaluated each day as described above.

Results from the above study demonstrate the construction of a set of complementary metabolizers that metabolize separate carbon sources in parallel at the substantial exclusion of the other. In particular, two strains of E. coli were engineered to grow on only one of the two carbon substrates, xylose and glucose, but not on the other. These results indicate: (i) that these CMO constituent strains, when cocultured, can consume both substrates simultaneously even though each of the strains can metabolize either glucose or xylose, and (ii) that the overall carbon utilization rate in the medium is enhanced in comparison to that of the unmodified wild-type E. coli MG1655, thus maximizing the productivity of a given fermentation. Each of the CMO constituent strains can be further designed to produce a desired chemical product such as succinic acid, for example.

Throughout this application various publications have been referenced within parentheses. The disclosures of these publications in their entireties are hereby incorporated by reference in this application in order to more fully describe the state of the art to which this invention pertains.

Although the invention has been described with reference to the disclosed embodiments, those skilled in the art will readily appreciate that the specific examples and studies detailed above are only illustrative of the invention. It should be understood that various modifications can be made without departing from the spirit of the invention. Accordingly, the invention is limited only by the following claims.

What is claimed is:

1. A non-naturally occurring set of microbial organisms, comprising:
at least a first constituent complementary metabolizing organism (CMO) exhibiting the ability to metabolize a first carbon substrate and having substantially impaired metabolic capacity for a second carbon substrate, and
at least a second constituent complementary metabolizing organism (CMO) exhibiting the ability to metabolize said second carbon substrate and having substantially impaired metabolic capacity for said first carbon substrate,
wherein a co-culture of said at least first and second CMOs exhibit simultaneous metabolism of a mixture comprising said first and second carbon substrates compared to either CMO alone.

2. The non-naturally occurring set of microbial organisms of claim 1, wherein said first carbon substrate comprises glucose, xylose, arabinose, galactose, mannose or fructose.

3. The non-naturally occurring set of microbial organisms of claim 1, wherein said second carbon substrate comprises glucose, xylose, arabinose, galactose, mannose or fructose.

4. The non-naturally occurring set of microbial organisms of claim 1, wherein said mixture comprising said first and second carbon substrates comprises a renewable feedstock.

5. The non-naturally occurring set of microbial organisms of claim 4, wherein said renewable feedstock comprises a biomass.

6. The non-naturally occurring set of microbial organisms of claim 5, wherein said renewable feedstock comprises a cellulosic biomass or a hemicellulosic biomass.

7. The non-naturally occurring set of microbial organisms of claim 6, wherein said renewable feedstock comprises a carbon source selected from carbohydrate, aromatic compounds or lignin.

8. The non-naturally occurring set of microbial organisms of claim 1, wherein said mixture comprising said first and second carbon substrates further comprises a toxic compound.

9. The non-naturally occurring set of microbial organisms of claim 1, wherein said first or second CMO's ability to metabolize a first or second carbon substrate, respectively, comprises an endogenous metabolic or substrate transport pathway.

10. The non-naturally occurring set of microbial organisms of claim 1, wherein said first or second CMO's ability to metabolize a first or second carbon substrate, respectively, comprises an exogenous metabolic or substrate transport pathway.

11. The non-naturally occurring set of microbial organisms of claim 1, wherein said impaired metabolic capacity for said second or first carbon substrate comprises an endogenous deficiency in a metabolic or substrate transport pathway.

12. The non-naturally occurring set of microbial organisms of claim 1, wherein said impaired metabolic capacity for said second or first carbon substrate comprises an exogenously introduced deficiency in a metabolic or substrate transport pathway.

13. The non-naturally occurring set of microbial organisms of claim 1, wherein at least one of said first or second constituent complementary metabolizing organisms further comprise one or more metabolic modifications encoding one or more enzymes conferring biosynthesis of a target chemical compound.

14. The non-naturally occurring set of microbial organisms of claim 13, further comprising each of said first or second constituent complementary metabolizing organisms having one or more metabolic modifications encoding one or more enzymes conferring biosynthesis of a target chemical compound.

15. The non-naturally occurring set of microbial organisms of claims 13 or 14 wherein said target chemical compound comprises succinic acid, fumaric acid, an isoprenoid, 3-hydroxypropionic acid or 1,4-butanediol

16. A bioprocess for producing a chemical compound, comprising co-culturing a non-naturally occurring set of microbial organisms in a mixture comprising at least a first and a second carbon substrate under conditions sufficient for biosynthesis of a target chemical compound, said set of non-naturally occurring microbial organisms comprising:
at least a first constituent complementary metabolizing organism (CMO) exhibiting the ability to metabolize said first carbon substrate and having substantially impaired metabolic capacity for said second carbon substrate, and
at least a second constituent complementary metabolizing organism (CMO) exhibiting the ability to metabolize said second carbon substrate and having substantially impaired metabolic capacity for said first carbon substrate,
wherein a co-culture of said at least first and second CMOs exhibit simultaneous metabolism of a mixture comprising said first and second carbon substrates compared to either CMO alone.

17. The bioprocess of claim 16, wherein said first carbon substrate comprises glucose, xylose, arabinose, galactose, mannose or fructose.

18. The bioprocess of claim 16, wherein said second carbon substrate comprises glucose, xylose, arabinose, galactose, mannose or fructose.

19. The bioprocess of claim 16, wherein said mixture comprising said first and second carbon substrates comprises a renewable feedstock.

20. The bioprocess of claim 19, wherein said renewable feedstock comprises a biomass.

21. The bioprocess of claim 20, wherein said renewable feedstock comprises a cellulosic biomass or a hemicellulosic biomass.

22. The bioprocess of claim 20, wherein said renewable feedstock comprises a carbon source selected from carbohydrate, aromatic compounds or lignin.

23. The bioprocess of claim 16, wherein said mixture comprising said first and second carbon substrates further comprises a toxic compound.

24. The bioprocess of claim 16, wherein said first or second CMO's ability to metabolize a first or second carbon substrate, respectively, comprises an endogenous metabolic or substrate transport pathway.

25. The bioprocess of claim 16, wherein said first or second CMO's ability to metabolize a first or second carbon substrate, respectively, comprises an exogenous metabolic or substrate transport pathway.

26. The bioprocess of claim 16, wherein said impaired metabolic capacity for said second or first carbon substrate comprises an endogenous deficiency in a metabolic or substrate transport pathway.

27. The bioprocess of claim 16, wherein said impaired metabolic capacity for said second or first carbon substrate comprises an exogenously introduced deficiency in a metabolic or substrate transport pathway.

28. The bioprocess of claim 16, wherein said co-culturing of said non-naturally occurring set of microbial organisms in a mixture comprises a process selected from batch fermentation, fed-batch fermentation or continuous fermentation.

29. The bioprocess of claim 16, wherein at least one of said first or second constituent complementary metabolizing organisms further comprise one or more metabolic modifications encoding one or more enzymes conferring biosynthesis of a target chemical compound.

30. The bioprocess of claim 29, further comprising each of said first or second constituent complementary metabolizing organisms having one or more metabolic modifications encoding one or more enzymes conferring biosynthesis of a target chemical compound.

31. The bioprocess of claims 29 or 30, wherein said target chemical compound comprises succinic acid, fumaric acid, an isoprenoid, 3-hydroxypropionic acid or 1,4-butanediol.
